(19) United States
(12) Patent Application Publication
(10) Pub. No.: US 2026/0029367 A1
(43) Pub. Date: Jan. 29, 2026

(54) GAS SENSOR

(71) Applicant: NGK INSULATORS, LTD., Nagoya-shi (JP)

(72) Inventor: Taku OKAMOTO, Nagoya-shi (JP)

(21) Appl. No.: 19/238,635

(22) Filed: Jun. 16, 2025

(30) Foreign Application Priority Data
Jul. 24, 2024 (JP) 2024-118632

(51) Int. Cl. G01N 27/41; G01N 27/406; G01N 27/407; G01N 33/00

(52) U.S. Cl. CPC G01N 27/41; G01N 27/4065; G01N 27/4072; G01N 33/0027

(57) ABSTRACT

A gas sensor includes a sensor element and a control unit for controlling the sensor element. The sensor element includes: a base part including an oxygen-ion-conductive solid electrolyte layer and a proton-conductive solid electrolyte layer, and having an insulator layer interposed between the oxygen-ion-conductive solid electrolyte layer and the proton-conductive solid electrolyte layer; an oxygen pump cell including an intracavity oxygen pump electrode disposed on the oxygen-ion-conductive solid electrolyte layer in an internal cavity; and a hydrogen pump cell including an intracavity hydrogen pump electrode disposed on the proton-conductive solid electrolyte layer in the internal cavity. The control unit includes a pump control part for controlling operation of the oxygen pump cell and the hydrogen pump cell; and a concentration calculating part for calculating a concentration of a target gas to be measured in a measurement-object gas.

### CROSS-REFERENCE TO RELATED APPLICATION

The present application claims priority from Japanese Patent Application No. 2024-118632, filed on Jul. 24, 2024, the content of which is hereby incorporated by reference into this application.

### BACKGROUND OF THE INVENTION

### Technical Field of the Invention

The present invention relates to a gas sensor including a sensor element using an ion-conductive solid electrolyte.

### Background Art

A gas sensor is used for detection or measurement of concentration of an objective gas component (oxygen O₂, nitrogen oxide NOx, ammonia NH₃, hydrocarbon HC, carbon dioxide CO₂, etc.) in a measurement-object gas, such as exhaust gas of automobile. As such a gas sensor, a gas sensor which has a sensor element using an oxygen ion conductive solid electrolyte such as zirconia (ZrO₂) is known.

JP 2020-067432 A discloses a carbon dioxide detection device that detects a concentration of carbon dioxide in consideration of an air fuel ratio and a water concentration, by using an ion conductor that conducts oxygen ions and a proton conductor that conducts hydrogen protons.

### CITATION LIST

### Patent Document

Patent Document 1: JP 2020-067432 A

### SUMMARY OF THE INVENTION

### Problems to be Solved by the Invention

JP 2020-067432 A discloses a sensor element having an ion conductor that conducts oxygen ions, a proton conductor that conducts hydrogen protons, and a gas chamber formed between the ion conductor and the proton conductor. In the sensor element, a standard gas duct facing the ion conductor, and a reference gas duct facing the proton conductor are arranged in addition to the gas chamber. In the sensor element having such a complex internal structure, a possible problem is that cracking is likely to occur due to thermal stress during temperature increase (heating) and decrease (cooling) of the sensor element. Another problem is that the sensor element requires many processes in its manufacture.

The present invention is made in consideration of the above problems, and it is an object of the present invention to provide a gas sensor that measures a target gas to be measured such as oxygen, water vapor and carbon dioxide in a measurement-object gas with high accuracy.

### Means for Solving the Problems

As a result of intensive studies, the present inventor reaches the present invention. The present invention includes the following aspects.
(1) A gas sensor for detecting a target gas to be measured in a measurement-object gas, the gas sensor comprising a sensor element and a control unit for controlling the sensor element, wherein
the sensor element comprises:
   a base part in an elongated plate shape, including an oxygen-ion-conductive solid electrolyte layer and a proton-conductive solid electrolyte layer, and having an insulator layer interposed between the oxygen-ion-conductive solid electrolyte layer and the proton-conductive solid electrolyte layer;
   a measurement-object gas flow cavity having a gas inlet that opens on a surface of the base part; and an internal cavity that communicates with the gas inlet via a first diffusion-rate limiting path, on an inner surface of the internal cavity the oxygen-ion-conductive solid electrolyte layer and the proton-conductive solid electrolyte layer being present;
   an oxygen pump cell including: an intracavity oxygen pump electrode disposed on the oxygen-ion-conductive solid electrolyte layer in the internal cavity of the measurement-object gas flow cavity; and an extracavity oxygen pump electrode disposed at a position different from the measurement-object gas flow cavity on the base part and adjacent to the intracavity oxygen pump electrode via the oxygen-ion-conductive solid electrolyte layer; and
   a hydrogen pump cell including: an intracavity hydrogen pump electrode disposed on the proton-conductive solid electrolyte layer in the internal cavity of the measurement-object gas flow cavity; and an extracavity hydrogen pump electrode disposed at a position different from the measurement-object gas flow cavity on the base part and adjacent to the intracavity hydrogen pump electrode via the proton-conductive solid electrolyte layer, and
the control unit comprises:
   a pump control part for controlling operation of the oxygen pump cell and the hydrogen pump cell; and
   a concentration calculating part for calculating a concentration of a target gas to be measured in a measurement-object gas, wherein
the pump control part applies a predetermined voltage between the intracavity oxygen pump electrode and the extracavity oxygen pump electrode of the oxygen pump cell to make an oxygen pump current flow through the oxygen pump cell; and applies a predetermined voltage between the intracavity hydrogen pump electrode and the extracavity hydrogen pump electrode of the hydrogen pump cell to make a hydrogen pump current flow through the hydrogen pump cell; and
the concentration calculating part calculates the concentration of the target gas to be measured in the measurement-object gas based on at least one of the oxygen pump current and the hydrogen pump current.
(2) The gas sensor according to the above (1), wherein the extracavity oxygen pump electrode and/or the extracavity hydrogen pump electrode are disposed at a position in contact with the measurement-object gas.
(3) The gas sensor according to the above (1) or (2), wherein the internal cavity has a first internal cavity on an inner surface of which at least the oxygen-ion-conductive solid electrolyte layer is present; and a second internal cavity that communicates with the first internal cavity via a second diffusion-rate limiting path, on an inner surface of the second internal cavity at least the proton-conductive solid electrolyte layer being present, and
the intracavity oxygen pump electrode is disposed in the first internal cavity and the intracavity hydrogen pump electrode is disposed in the second internal cavity.
(4) The gas sensor according to any one of the above (1) to (3), wherein
the sensor element further comprises:
   an oxidizing pump cell including: an intracavity oxidizing electrode disposed at a position farther from the first diffusion-rate limiting path than the intracavity oxygen pump electrode on the oxygen-ion-conductive solid electrolyte layer in the internal cavity of the measurement-object gas flow cavity; and an extracavity oxidizing electrode disposed at a position different from the measurement-object gas flow cavity on the base part and adjacent to the intracavity oxidizing electrode via the oxygen-ion-conductive solid electrolyte layer,
the pump control part further applies a predetermined voltage between the intracavity oxidizing electrode and the extracavity oxidizing electrode of the oxidizing pump cell to make an oxidizing pump current flow through the oxidizing pump cell, and
the concentration calculating part calculates the concentration of the target gas to be measured in the measurement-object gas based on at least one of the oxygen pump current, the hydrogen pump current, and the oxidizing pump current.
(5) The gas sensor according to the above (4), wherein the intracavity oxidizing electrode is disposed on the oxygen-ion-conductive solid electrolyte layer at a position farther from the first diffusion-rate limiting path than the intracavity hydrogen pump electrode.
(6) The gas sensor according to the above (4) or (5), wherein the internal cavity has a first internal cavity on an inner surface of which at least the oxygen-ion-conductive solid electrolyte layer is present; and a second internal cavity that communicates with the first internal cavity via a second diffusion-rate limiting path, on an inner surface of the second internal cavity the oxygen-ion-conductive solid electrolyte layer and the proton-conductive solid electrolyte layer being present, and
the intracavity oxygen pump electrode is disposed in the first internal cavity, the intracavity hydrogen pump electrode is disposed on the proton-conductive solid electrolyte layer in the second internal cavity, and the intracavity oxidizing electrode is disposed on the oxygen-ion-conductive solid electrolyte layer in the second internal cavity.
(7) The gas sensor according to the above (4) or (5), wherein the internal cavity has a first internal cavity on an inner surface of which at least the oxygen-ion-conductive solid electrolyte layer is present; a second internal cavity that communicates with the first internal cavity via a second diffusion-rate limiting path, on an inner surface of the second internal cavity at least the proton-conductive solid electrolyte layer being present; and a third internal cavity that communicates with the second internal cavity via a third diffusion-rate limiting path, on an inner surface of the third internal cavity at least the oxygen-ion-conductive solid electrolyte layer being present, and
the intracavity oxygen pump electrode is disposed in the first internal cavity, the intracavity hydrogen pump electrode is disposed in the second internal cavity, and the intracavity oxidizing electrode is disposed in the third internal cavity.
(8) The gas sensor according to any one of the above (1) to (7), wherein
the sensor element comprises a reference electrode disposed inside the base part to be in contact with a reference gas, and
the pump control part applies the predetermined voltage between the intracavity oxygen pump electrode and the extracavity oxygen pump electrode of the oxygen pump cell based on an electromotive force between the reference electrode and the intracavity oxygen pump electrode of the oxygen pump cell to make the oxygen pump current flow through the oxygen pump cell.

The gas sensor according to any one of the above (4) to (7), wherein
the sensor element comprises a reference electrode disposed inside the base part to be in contact with a reference gas, and
the pump control part applies the predetermined voltage between the intracavity oxygen pump electrode and the extracavity oxygen pump electrode of the oxygen pump cell based on an electromotive force between the reference electrode and the intracavity oxygen pump electrode of the oxygen pump cell to make the oxygen pump current flow through the oxygen pump cell; and applies the predetermined voltage between the intracavity oxidizing electrode and the extracavity oxidizing electrode of the oxidizing pump cell based on an electromotive force between the reference electrode and the intracavity oxidizing electrode of the oxidizing pump cell to make the oxidizing pump current flow through the oxidizing pump cell.
(9) The gas sensor according to any one of the above (1) to (8), wherein, in a plano including a longitudinal direction of the base part and a width direction perpendicular to the longitudinal direction, the proton-conductive solid electrolyte layer and the oxygen-ion-conductive solid electrolyte layer are present on the same plane, and the insulator layer is interposed between the oxygen-ion-conductive solid electrolyte layer and the proton-conductive solid electrolyte layer.
(10) The gas sensor according to any one of the above (1) to (8), wherein
the oxygen-ion-conductive solid electrolyte layer has a penetrating hole,
the proton-conductive solid electrolyte layer is parallel to the oxygen-ion-conductive solid electrolyte layer, and covers the penetrating hole on a surface of the oxygen-ion-conductive solid electrolyte layer facing the internal cavity; and the insulator layer is interposed between the proton-conductive solid electrolyte layer and the oxygen-ion-conductive solid electrolyte layer,
the intracavity hydrogen pump electrode is disposed on a surface of the proton-conductive solid electrolyte layer facing the internal cavity, and
the extracavity hydrogen pump electrode is disposed at a position corresponding to the penetrating hole on a surface of the proton-conductive solid electrolyte layer opposite to the surface on which the intracavity hydrogen pump electrode is disposed.
(11) The gas sensor according to any one of the above (1) to (3), wherein the target gas to be measured is at least one selected from the group consisting of oxygen and water vapor.
(12) The gas sensor according to any one of the above (4) to (10), wherein the target gas to be measured is at least one selected from the group consisting of oxygen, water vapor and carbon dioxide.

### Advantageous Effect of the Invention

According to the present invention, it is possible to provide a gas sensor that measures a target gas to be measured such as oxygen, water vapor and carbon dioxide in a measurement-object gas with high accuracy.

### BRIEF DESCRIPTION OF THE DRAWINGS

FIG. 1 is a vertical sectional schematic view in the longitudinal direction of a sensor element 101, showing one example of a schematic configuration of a gas sensor 100 of Embodiment 1.

FIG. 2 is a partial sectional schematic view in the same section of FIG. 1, showing an arrangement of a measurement-object gas flow part 14 and its surrounding in the sensor element 101.

FIG. 3 is a block diagram showing electric connections between a control unit 90 and respective pump cells 21 and 31 of the sensor element 101.

FIG. 4 is a vertical sectional schematic view in the longitudinal direction of a sensor element 111, showing one example of a schematic configuration of a gas sensor 110.

FIG. 5 is a vertical sectional schematic view in the longitudinal direction of a sensor element 201, showing one example of a schematic configuration of a gas sensor 200 of Embodiment 2.

FIG. 6 is a partial sectional schematic view in the same section of FIG. 5, showing an arrangement of a measurement-object gas flow part 15 and its surrounding in the sensor element 201.

FIG. 7 is a block diagram showing electric connections between a control unit 290 and respective pump cells 21, 31 and 51 of the sensor element 201 in the gas sensor 200 of Embodiment 2.

FIG. 8 is a partial sectional schematic view in the same section of FIG. 5, showing another example of arrangement of a measurement-object gas flow part and its surrounding.

FIG. 9 is a partial sectional schematic view in the same section of FIG. 5, showing another example of arrangement of a measurement-object gas flow part and its surrounding.

FIG. 10 is a vertical sectional schematic view in the longitudinal direction of a sensor element 301, showing one example of a schematic configuration of a gas sensor 300 of Embodiment 3.

FIG. 11 is a block diagram showing electric connections between a control unit 390 and respective pump cells 21 and 31 and an oxygen pump controlling sensor cell 80 of the sensor element 301 in the gas sensor 300 of Embodiment 3.

FIG. 12 is a vertical sectional schematic view in the longitudinal direction of a sensor element 401, showing one example of a schematic configuration of a gas sensor 400 of Embodiment 4.

FIG. 13 is a block diagram showing electric connections between a control unit 490 and respective pump cells 21, 31 and 51 and respective sensor cells 80 and 81 of the sensor element 401 in the gas sensor 400 of Embodiment 4.

FIG. 14 is a partial sectional schematic view in the same section of FIG. 5, showing another example of arrangement of a measurement-object gas flow part and its surrounding.

FIG. 15 is a graph showing a relationship between the oxygen concentration (O₂ concentration) in the measurement-object gas and each of the pump current Ip0, Ip1 and Ip2. The horizontal axis of the graph represents O₂ concentration (%), and the vertical axis represents a current value (μA) of each of pump currents Ip0, Ip1, Ip2. The primary axis of the vertical axis represents the pump current Ip0, and the secondary axis of the vertical axis represents the pump currents Ip1 and Ip2.

FIG. 16 is a graph showing a relationship between the water vapor concentration (H₂O concentration) in the measurement-object gas and each of the pump current Ip0, Ip1 and Ip2. The horizontal axis of the graph represents H₂O concentration (%), and the vertical axis represents a current value (μA) of each of pump currents Ip0, Ip1, Ip2. The primary axis of the vertical axis represents the pump current Ip0, and the secondary axis of the vertical axis represents the pump currents Ip1 and Ip2.

FIG. 17 is a graph showing a relationship between the carbon dioxide concentration (CO₂ concentration) in the measurement-object gas and each of the pump current Ip0, Ip1 and Ip2. The horizontal axis of the graph represents CO₂ concentration (%), and the vertical axis represents a current value (μA) of each of pump currents Ip0, Ip1, Ip2. The primary axis of the vertical axis represents the pump current Ip0, and the secondary axis of the vertical axis represents the pump currents Ip1 and Ip2.

### MODES FOR CARRYING OUT OF THE INVENTION

A gas sensor of the present invention includes a sensor element and a control unit for controlling the sensor element.

The sensor element contained in the gas sensor of the present invention includes:
a base part in an elongated plate shape, including an oxygen-ion-conductive solid electrolyte layer and a proton-conductive solid electrolyte layer, and having an insulator layer interposed between the oxygen-ion-conductive solid electrolyte layer and the proton-conductive solid electrolyte layer;
a measurement-object gas flow cavity having a gas inlet that opens on a surface of the base part; and an internal cavity that communicates with the gas inlet via a first diffusion-rate limiting path, on an inner surface of the internal cavity the oxygen-ion-conductive solid electrolyte layer and the proton-conductive solid electrolyte layer being present;
an oxygen pump cell including: an intracavity oxygen pump electrode disposed on the oxygen-ion-conductive solid electrolyte layer in the internal cavity of the measurement-object gas flow cavity; and an extracavity oxygen pump electrode disposed at a position different from the measurement-object gas flow cavity on the base part and adjacent to the intracavity oxygen pump electrode via the oxygen-ion-conductive solid electrolyte layer; and
a hydrogen pump cell including: an intracavity hydrogen pump electrode disposed on the proton-conductive solid electrolyte layer in the internal cavity of the measurement-object gas flow cavity; and an extracavity hydrogen pump electrode disposed at a position different from the measurement-object gas flow cavity on the base part and adjacent to the intracavity hydrogen pump electrode via the proton-conductive solid electrolyte layer.

The control unit contained in the gas sensor of the present invention includes
a pump control part for controlling operation of the oxygen pump cell and the hydrogen pump cell; and
a concentration calculating part for calculating a concentration of a target gas to be measured in a measurement-object gas, wherein
the pump control part applies a predetermined voltage between the intracavity oxygen pump electrode and the extracavity oxygen pump electrode of the oxygen pump cell to make an oxygen pump current flow through the oxygen pump cell; and applies a predetermined voltage between the intracavity hydrogen pump electrode and the extracavity hydrogen pump electrode of the hydrogen pump cell to make a hydrogen pump current flow through the hydrogen pump cell; and
the concentration calculating part calculates the concentration of the target gas to be measured in the measurement-object gas based on at least one of the oxygen pump current and the hydrogen pump current.

### Embodiment 1

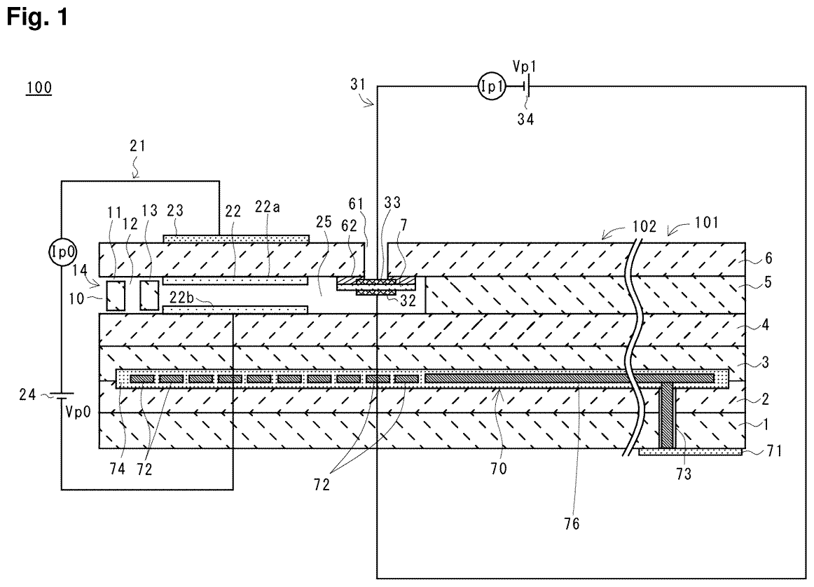

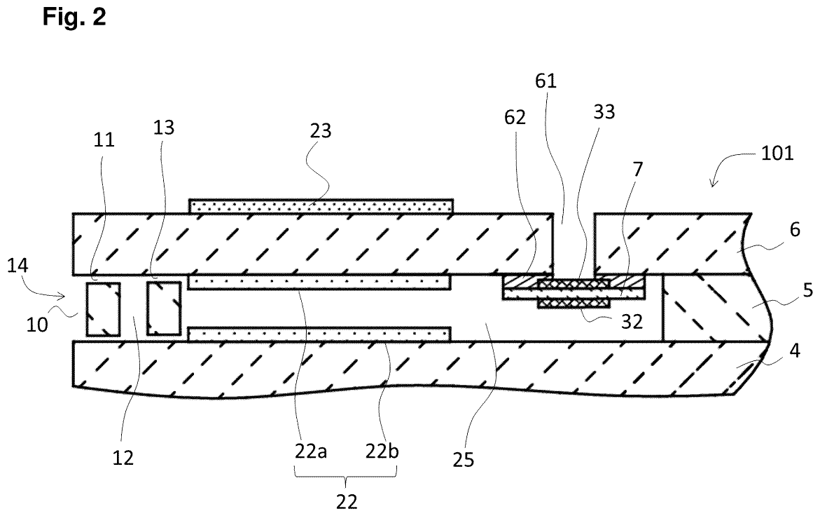

One example of embodiments of the gas sensor of the present invention will now be described with reference to the drawings. FIG. 1 is a vertical sectional schematic view in the longitudinal direction, showing one example of a schematic configuration of a gas sensor 100 including a sensor element 101. Hereinafter, based on FIG. 1, the upper side and the lower side in FIG. 1 are respectively defined as top and bottom, and the left side and the right side in FIG. 1 are respectively defined as a front end side and a rear end side. FIG. 2 is a partial sectional schematic view in the same section of FIG. 1, showing an arrangement of a measurement-object gas flow part 14 and its surrounding in the sensor element 101.

In FIG. 1, the gas sensor 100 represents one example of a gas sensor that detects oxygen O₂ and water vapor H₂O in a measurement-object gas by the sensor element 101, and measures the concentrations of O₂ and H₂O.

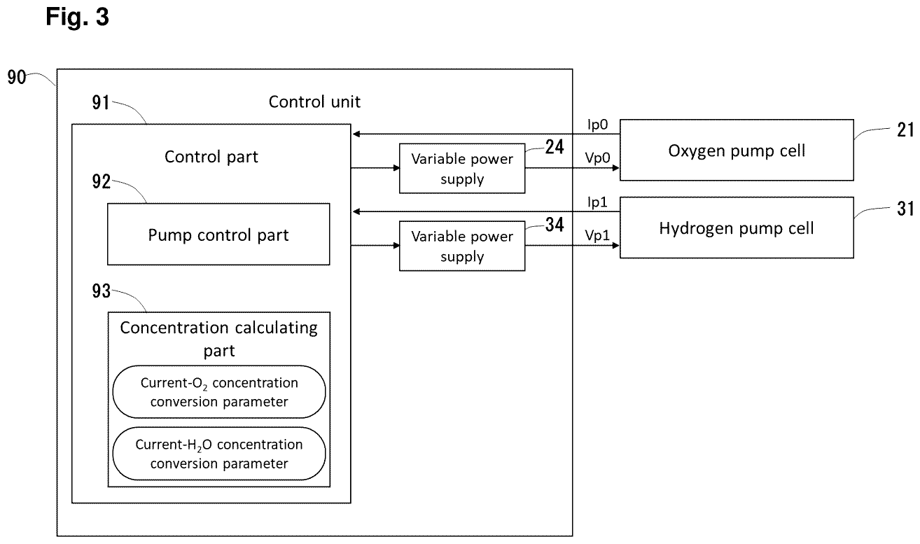

Further, the gas sensor 100 includes a control unit 90 for controlling the sensor element 101. FIG. 3 is a block diagram showing electric connections between the control unit 90 and the sensor element 101.

### (Sensor Element)

The sensor element 101 is an element in an elongated plate shape, including a base part 102 having an oxygen-ion-conductive solid electrolyte layer (in this embodiment, a second oxygen-ion conductor layer 6) and a proton-conductive solid electrolyte layer (in this embodiment, a proton conductor layer 7). The elongated plate shape also called a long plate shape or a belt shape.

The oxygen-ion-conductive solid electrolyte layer is formed of an oxygen-ion-conductive solid electrolyte (namely, an oxygen-ion conductor), and extends in a longitudinal direction of the sensor element 101 (the base part 102). As the oxygen-ion-conductive solid electrolyte (the oxygen-ion conductor), for example, stabilized zirconia and partially stabilized zirconia, in which a rare earth metal oxide or an alkaline earth metal oxide is added to zirconia as a stabilizing agent, may be used. Examples of the stabilizing agents include yttria (Y₂O₃), calcia (CaO), magnesia (MgO), ceria (CeO₂), and scandia (Sc₂O₃). For example, yttria-stabilized zirconia may be used.

The proton-conductive solid electrolyte layer is formed of a proton-conductive solid electrolyte (namely, a proton conductor), and extends in the longitudinal direction of the sensor element 101 (the base part 102). As the proton-conductive solid electrolyte (the proton conductor), for example, perovskite type oxide and the like may be used. As the proton-conductive solid electrolyte (the proton conductor), for example, a perovskite type ceramic represented by the following composition formula may be used.

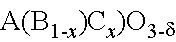

Here, “A” is, for example, a bivalent metal selected from the group consisting of Ba, Ca, and Sr. “B” is, for example, a tetravalent metal selected from the group consisting of Ce and Zr. “C” is, for example, a trivalent metal selected from the group consisting of In, Y, Yb, Mn, and Sc. “C” is a so-called dopant. “x” may be 0 or more and 0.7 or less. Specifically, CaZr_{0.9} In_{0.1} O₃₋₈ and SrZr_{0.9} Y_{0.1} O₃₋₈ are exemplified.

The base part 102 has such a structure that six layers, namely, a first substrate layer 1, a second substrate layer 2, a third substrate layer 3, a first oxygen-ion conductor layer 4, a spacer layer 5, and a second oxygen-ion conductor layer 6, are layered in substantially parallel in this order from the bottom side, as viewed in the drawing. Each of the six layers is formed of an oxygen-ion-conductive solid electrolyte layer containing, for example, zirconia (ZrO₂). The solid electrolyte forming these six layers is dense and gastight. These six layers all may have the same thickness, or the thickness may vary among the layers. The layers are adhered to each other with an adhesive layer of a solid electrolyte interposed therebetween, and the base part 102 includes the adhesive layer. While a layer configuration composed of the six layers is illustrated in FIG. 1, the layer configuration in the present invention is not limited to this, and any number of layers and any layer configuration are possible. Further, a part of the six layers (for example, the first substrate layer 1, the second substrate layer 2, and the third substrate layer 3) may be composed of, for example, a dense layer formed of an insulator such as alumina.

A penetrating hole 61 is formed in the second oxygen-ion conductor layer 6. The penetrating hole 61 penetrates the second oxygen-ion conductor layer 6 in the vertical direction of FIG. 1, that is, in a thickness direction of the second oxygen-ion conductor layer 6. At a position where the penetrating hole 61 exists, a proton conductor layer 7 is disposed on a lower surface of the second oxygen-ion conductor layer 6 via an insulator layer 62. The insulator layer 62 is a dense layer formed of an insulator such as alumina. The insulator layer 62 is interposed between the second oxygen-ion conductor layer 6 and the proton conductor layer 7 to ensure electrical insulation between the second oxygen-ion conductor layer 6 having oxygen-ion conductivity and the proton conductor layer 7 having proton conductivity.

A measurement-object gas flow cavity 14 has a gas inlet 10 that opens on a surface of the base part 102, and an internal cavity 25 that communicates with the gas inlet 10 via a first diffusion-rate limiting path 11 (namely, a first diffusion-rate limiting part), on an inner surface of the internal cavity 25 the oxygen-ion-conductive solid electrolyte layer (namely, the second oxygen-ion conductor layer 6 and the first oxygen-ion conductor layer 4) and the proton-conductive solid electrolyte layer (namely, the proton conductor layer 7) being present. That is, Embodiment 1 is an example of a structure having one internal cavity.

The gas inlet 10 is formed between the lower surface of the second oxygen-ion conductor layer 6 and the upper surface of the first oxygen-ion conductor layer 4 in one end part in the longitudinal direction (hereinafter, referred to as a front end part) of the sensor element 101. The measurement-object gas flow cavity 14, that is, a measurement-object gas flow part is formed in such a form that the first diffusion-rate limiting path 11, a buffer space 12, a fourth diffusion-rate limiting path 13 (namely, a fourth diffusion-rate limiting part), and the internal cavity 25 communicate in this order in the longitudinal direction from the gas inlet 10. The internal cavity 25 faces the first oxygen-ion conductor layer 4, the second oxygen-ion conductor layer 6, and the proton conductor layer 7.

The gas inlet 10, the buffer space 12, and the internal cavity 25 constitute internal spaces of the sensor element 101. Each of the internal spaces is provided in such a manner that a portion of the spacer layer 5 is hollowed out, and the top of each of the internal spaces is defined by the lower surface of the second oxygen-ion conductor layer 6, the bottom of each of the internal spaces is defined by the upper surface of the first oxygen-ion conductor layer 4, and the lateral surface of each of the internal spaces is defined by the lateral surface of the spacer layer 5.

Each of the first diffusion-rate limiting path 11 and the fourth diffusion-rate limiting path 13 is provided as two laterally elongated slits (having the longitudinal direction of the openings in the direction perpendicular to the figure in FIG. 1). Each of the first diffusion-rate limiting path 11 and the fourth diffusion-rate limiting path 13 may be in such a form that a desired diffusion resistance is created, but the form is not limited to the slits.

In the measurement-object gas flow cavity 14, the gas inlet 10 is open to the external space, and the measurement-object gas is taken into the sensor element 101 from the external space through the gas inlet 10.

In the present embodiment, the measurement-object gas flow cavity 14 is in such a form that the measurement-object gas is introduced through the gas inlet 10 that is open on the front end surface of the sensor element 101, however, the present invention is not limited to this form. For example, the measurement-object gas flow cavity 14 need not have a recess of the gas inlet 10. In this case, the first diffusion-rate limiting path 11 substantially serves as a gas inlet.

For example, the measurement-object gas flow cavity 14 may have an opening that communicates with the buffer space 12 or a position near the buffer space 12 of the internal cavity 25, on a lateral surface along the longitudinal direction of the base part 102. In this case, the measurement-object gas is introduced from the lateral surface along the longitudinal direction of the base part 102 through the opening.

Further, for example, the measurement-object gas flow cavity 14 may be so configured that the measurement-object gas is introduced through a porous body.

The first diffusion-rate limiting path 11 creates a predetermined diffusion resistance to the measurement-object gas taken through the gas inlet 10.

The buffer space 12 is provided to mitigate the influence of pressure fluctuation on the detected value when the pressure of the measurement-object gas fluctuates. The sensor element 101 may have a structure without the buffer space 12.

The fourth diffusion-rate limiting path 13 creates a predetermined diffusion resistance to the measurement-object gas introduced into the internal cavity 25 from the buffer space 12. The fourth diffusion-rate limiting path 13 is provided in association with the buffer space 12 being provided.

When the buffer space 12 and the fourth diffusion-rate limiting path 13 are not provided, the first diffusion-rate limiting path 11 directly communicates with the internal cavity 25.

The internal cavity 25 is provided as a space for measuring oxygen concentration and water vapor concentration in the measurement-object gas introduced through the fourth diffusion-rate limiting path 13. These concentrations are measured by the operation of an oxygen pump cell 21 and a hydrogen pump cell 31.

The oxygen pump cell 21 is an electrochemical pump cell including an intra-cavity oxygen pump electrode (in this embodiment, an inner oxygen pump electrode 22) disposed on the oxygen-ion-conductive solid electrolyte layer (in this embodiment, the first oxygen-ion conductor layer 4 and the second oxygen-ion conductor layer 6) in the internal cavity 25 of the measurement-object gas flow cavity 14; and an extra-cavity oxygen pump electrode (in this embodiment, an outer oxygen pump electrode 23) disposed at a position different from the measurement-object gas flow cavity 14 on the base part 102 and adjacent to the inner oxygen pump electrode 22 via the second oxygen-ion conductor layer 6.

That is, the oxygen pump cell 21 is an electrochemical pump cell composed of the inner oxygen pump electrode 22 having a ceiling electrode portion 22a disposed on the lower surface of the second oxygen-ion conductor layer 6 that faces the internal cavity 25, the outer oxygen pump electrode 23 disposed on a region of the upper surface of the second oxygen-ion conductor layer 6 that corresponds to the ceiling electrode portion 22a so as to be exposed to the external space, and the second oxygen-ion conductor layer 6 sandwiched between the inner oxygen pump electrode 22 and the outer oxygen pump electrode 23.

The inner oxygen pump electrode 22 is formed to span the upper and lower solid electrolyte layers (the second oxygen-ion conductor layer 6 and the first oxygen-ion conductor layer 4) that define the internal cavity 25 and the spacer layer 5 that defines the lateral wall. Specifically, the ceiling electrode portion 22a is formed on the lower surface of the second oxygen-ion conductor layer 6 that defines the ceiling surface of the internal cavity 25, and a bottom electrode portion 22b is formed on the upper surface of the first oxygen-ion conductor layer 4 that defines the bottom surface of the internal cavity 25. Also, lateral electrode portions (not shown) are formed on the lateral wall surfaces (inner surface) of the spacer layer 5 that form both lateral wall parts of the internal cavity 25 so as to connect the ceiling electrode portion 22a and the bottom electrode portion 22b. Thus, the inner oxygen pump electrode 22 is provided as a tunnel-like structure in the area where the lateral electrode portions are disposed. The inner oxygen pump electrode 22 exists between an inner surface of the internal cavity 25 in the measurement-object gas flow cavity 14 and a space of the internal cavity 25. The inner oxygen pump electrode 22 may be disposed on one of the ceiling surface and the bottom surface in the internal cavity 25.

The inner oxygen pump electrode 22 and the outer oxygen pump electrode 23 are porous cermet electrodes (electrodes in a state that a metal component and a ceramic component are mixed). The ceramic component to be used is not particularly limited, but is preferably an oxygen-ion-conductive solid electrolyte as in the case of the second oxygen-ion conductor layer 6. For example, ZrO₂ (stabilized ZrO₂) can be used as the ceramic component.

The inner oxygen pump electrode 22 and the outer oxygen pump electrode 23 preferably contain a noble metal having catalytic activity (e.g., at least one of Pt, Rh, Ir, Ru, and Pd) as the metal component. For example, the inner oxygen pump electrode 22 and the outer oxygen pump electrode 23 may be porous cermet electrodes made of Pt and ZrO₂.

In the oxygen pump cell 21, a desired pump voltage Vp0 is applied between the inner oxygen pump electrode 22 and the outer oxygen pump electrode 23 by a variable power supply 24 to make an oxygen pump current Ip0 flow between the inner oxygen pump electrode 22 and the outer oxygen pump electrode 23, and thus it is possible to pump out oxygen in the internal cavity 25 to the external space. At this time, it is also possible to decompose water vapor in the measurement-object gas and pump out oxygen generated by the decomposition of the water vapor.

The above-described penetrating hole 61 exists at a position farther from the first diffusion-rate limiting path 11 than the inner oxygen pump electrode 22 in the second oxygen-ion conductor layer 6. Size and a shape of the penetrating hole 61 is not particularly limited. The penetrating hole 61 may exist on at least a part of a region where the inner oxygen pump electrode 22 does not exist on the ceiling surface of the internal cavity 25, and the shape of the opening of the penetrating hole 61 in a planar view may be a circular shape, an elliptical shape, a rectangular shape or the like. As shown in FIG. 2, the proton conductor layer 7 is substantially parallel to the second oxygen-ion conductor layer 6, and exists at a position where the penetrating hole 61 is covered on the lower surface of the second oxygen-ion conductor layer 6, that is, on the surface facing the internal cavity 25. The proton conductor layer 7 and the second oxygen-ion conductor layer 6 may be formed as separate layers. The proton conductor layer 7 and the second oxygen-ion conductor layer 6 are normally located in substantially parallel, but may not be in parallel. The insulator layer 62 is interposed between the proton conductor layer 7 and the second oxygen-ion conductor layer 6 so that the proton conductor layer 7 and the second oxygen-ion conductor layer 6 do not directly contact each other. As described above, the insulator layer 62 is the dense layer formed of the insulator such as alumina. The insulator layer 62 is not formed at a position corresponding to the penetrating hole 61. Therefore, an upper surface of the proton conductor layer 7 faces outside of the sensor element 101 at a position corresponding to the penetrating hole 61. The second oxygen-ion conductor layer 6, the insulator layer 62, and the proton conductor layer 7 are all dense layers, and contact closely to each other. Accordingly, the measurement-object gas is introduced into the internal cavity 25 through the gas inlet 10.

The hydrogen pump cell 31 is an electrochemical pump cell including an intra-cavity hydrogen pump electrode (in this embodiment, an inner hydrogen pump electrode 32) disposed on the proton conductor layer 7 in the internal cavity 25 of the measurement-object gas flow cavity 14; and an extra-cavity hydrogen pump electrode (in this embodiment, an outer hydrogen pump electrode 33) disposed at a position different from the measurement-object gas flow cavity 14 on the base part 102 and adjacent to the inner hydrogen pump electrode 32 via the proton conductor layer 7.

That is, referring to FIG. 1 and FIG. 2, the hydrogen pump cell 31 is an electrochemical pump cell composed of the inner hydrogen pump electrode 32 disposed on the lower surface of the proton conductor layer 7 that faces the internal cavity 25, the outer hydrogen pump electrode 33 disposed on a region of the upper surface of the proton conductor layer 7 that corresponds to the penetrating hole 61 so as to be exposed to the external space, and the second proton conductor layer 7 sandwiched between the inner hydrogen pump electrode 32 and the outer hydrogen pump electrode 33. The outer hydrogen pump electrode 33 is in contact with the measurement-object gas as in the case of the outer oxygen pump electrode 23.

In FIG. 1 and FIG. 2, the outer hydrogen pump electrode 33 is larger than the size of opening of the penetrating hole 61, and thus the outer hydrogen pump electrode 33 is exposed over the entire surface at the innermost of the penetrating hole 61 when the penetrating hole 61 is viewed from above. The planar shape of the outer hydrogen pump electrode 33 may be substantially the same as the opening shape of the penetrating hole 61. Alternatively, the planar shape of the outer hydrogen pump electrode 33 may be larger or smaller than the opening of the penetrating hole 61. When the planar shape of the outer hydrogen pump electrode 33 is substantially the same as, or larger than the opening of the penetrating hole 61, the outer hydrogen pump electrode 33 is exposed over the entire surface at the innermost of the penetrating hole 61 when the penetrating hole 61 is viewed from above. When the planar shape of the outer hydrogen pump electrode 33 is smaller than the opening of the penetrating hole 61, the outer hydrogen pump electrode 33 and the proton conductor layer 7 are exposed at the innermost of the penetrating hole 61 when the penetrating hole 61 is viewed from above. In all cases, the outer hydrogen pump electrode 33 has a portion exposed to the external space via the penetrating hole 61.

The inner hydrogen pump electrode 32 and the outer hydrogen pump electrode 33 are porous cermet electrodes (electrodes in a state that a metal component and a ceramic component are mixed). The ceramic component to be used is not particularly limited, but is preferably an hydrogen-ion (proton) conductive solid electrolyte as in the case of the proton conductor layer 7. For example, CaZr_{0.9} In_{0.1} O₃₋₈, SrZr_{0.9} Y_{0.1} O₃₋₈ and the like can be used as the ceramic component.

The inner hydrogen pump electrode 32 and the outer hydrogen pump electrode 33 preferably contain a noble metal having catalytic activity (e.g., at least one of Pt, Rh, Ir, Ru, and Pd) as the metal component. For example, the inner hydrogen pump electrode 32 and the outer hydrogen pump electrode 33 may be porous cermet electrodes made of Pt and ceramics such as CaZr_{0.9} In_{0.1} O₃₋₈ and SrZr_{0.9} Y_{0.1} O₃₋₈.

In the hydrogen pump cell 31, a desired pump voltage Vp1 is applied between the inner hydrogen pump electrode 32 and the outer hydrogen pump electrode 33 by a variable power supply 34 to make a hydrogen pump current Ip1 flow between the inner hydrogen pump electrode 32 and the outer hydrogen pump electrode 33, and thus it is possible to pump out hydrogen generated by the decomposition of the water vapor in the inner oxygen pump electrode 22 to the external space.

The sensor element 101 further includes a heater part 70 that functions as a temperature regulator of heating and maintaining the temperature of the sensor element 101 so as to enhance the oxygen ion conductivity or the hydrogen ion conductivity of the solid electrolytes. The heater part 70 includes a heater electrode 71, a heater 72, a through hole 73, a heater lead 76, and a heater insulating layer 74.

The heater electrode 71 is an electrode formed in contact with the lower surface of the first substrate layer 1. The power can be supplied to the heater part 70 from the outside by connecting the heater electrode 71 with an external power supply.

The heater 72 is an electrical resistor sandwiched by the second substrate layer 2 and the third substrate layer 3 from top and bottom. The heater 72 is connected with the heater electrode 71 via the heater lead 76 that connects with the heater 72 and extends in the rear end side in the longitudinal direction of the sensor element 101, and the through hole 73. The heater 72 is externally powered by a heater power supply 77 through the heater electrode 71 to generate heat, and heats and maintains the temperature of the solid electrolyte forming the sensor element 101.

The heater 72 is embedded over the whole area over the internal cavity 25 so that the temperature of the entire sensor element 101 can be adjusted to such a temperature that activates the solid electrolyte (both of the oxygen-ion-conductive solid electrolyte and the hydrogen-ion-conductive solid electrolyte). The temperature may be adjusted so that the oxygen pump cell 21 and the hydrogen pump cell 31 are operable. It is not necessary that the whole area is adjusted to the same temperature, but the sensor element 101 may have temperature distribution. By maintaining the heater 72 at a desired temperature, the sensor element 101 can be maintained at a driving temperature (e.g. about 800° C.) at which the solid electrolyte is activated and thus oxygen concentration and H₂O concentration are accurately measured.

In the sensor element 101 of the present embodiment, the heater 72 is embedded in the base part 102, but this form is not limitative. The heater 72 may be disposed to heat the base part 102. That is, the heater 72 may heat the sensor element 101 to develop oxygen ion conductivity with which the oxygen pump cell 21 is operable, and to develop hydrogen ion conductivity with which the hydrogen pump cell 31 is operable. For example, the heater 72 may be embedded in the base part 102 as in the present embodiment. Alternatively, for example, the heater part 70 may be formed as a heater substrate that is separate from the base part 102, and may be disposed at a position adjacent to the base part 102.

The heater insulating layer 74 is formed of an insulator such as alumina on the upper and lower surfaces of the heater 72 and the heater lead 76. The heater insulating layer 74 is formed to ensure electrical insulation between the second substrate layer 2, and the heater 72 and the heater lead 76, and electrical insulation between the third substrate layer 3, and the heater 72 and the heater lead 76.

### (Control Unit)

The gas sensor 100 of this embodiment includes the sensor element 101 described above and the control unit 90 for controlling the sensor element 101. In the gas sensor 100, each of the electrodes 22, 23, 32, and 33 of the sensor element 101 is electrically connected to the control unit 90 through a lead wire not shown. FIG. 3 is a block diagram showing electric connections between the control unit 90 and the respective pump cells 21 and 31 of the sensor element 101. The control unit 90 includes the above-described variable power supplies 24 and 34 and a control part 91. The control part 91 includes a pump control part 92 and a concentration calculating part 93.

The control part 91 is realized by a general-purpose or dedicated computer, and functions as the pump control part 92 and the concentration calculating part 93 are realized by a CPU, a memory or the like installed in the computer. It is to be noted that when oxygen and water vapor in exhaust gas from the engine of a car is target gases to be measured by the gas sensor 100 and the sensor element 101 is attached to an exhaust gas path, some or all of the functions of the control unit 90 (especially, the control unit 91) may be realized by an electronic control unit (ECU) installed in the car.

The control part 91 is configured to acquire a pump current (Ip0, Ip1) in each of the pump cells 21 and 31 of the sensor element 101. Further, the control part 91 is configured to output control signals to the variable power supplies 24 and 34.

The pump control part 92 is configured to control the operation of the oxygen pump cell 21 and the hydrogen pump cell 31 so as to measure a concentration of each of target gases to be measured (in this embodiment, oxygen and water vapor) in a measurement-object gas.

The pump control part 92 applies a predetermined pump voltage Vp0 between the intracavity oxygen pump electrode (the inner oxygen pump electrode 22) and the extracavity oxygen pump electrode (the outer oxygen pump electrode 23) in the oxygen pump cell 21 to make an oxygen pump current Ip0 flow through the oxygen pump cell 21; and applies a predetermined pump voltage Vp1 between the intracavity hydrogen pump electrode (the inner hydrogen pump electrode 32) and the extracavity hydrogen pump electrode (the outer hydrogen pump electrode 33) in the hydrogen pump cell 31 to make a hydrogen pump current Ip1 flow through the hydrogen pump cell 31.

The pump control part 92 applies the predetermined pump voltage Vp0 between the intracavity oxygen pump electrode (the inner oxygen pump electrode 22) and the extracavity oxygen pump electrode (the outer oxygen pump electrode 23) by the variable power supply 24 to pump out oxygen in the measurement-object gas from the internal cavity 25. At this time, water vapor in the measurement-object gas is decomposed (or, reduced) at the inner oxygen pump electrode 22, and both of oxygen generated by the decomposition of the water vapor and oxygen originally existing in the measurement-object gas are pumped out from the internal cavity 25. In this case, the oxygen pump current Ip0 flowing through the oxygen pump cell 21 flows from the inner oxygen pump electrode 22 toward the outer oxygen pump electrode 23 in the outside of the sensor element 101. The oxygen pump current Ip0 includes a current that flows due to the oxygen gas originally exiting in the measurement-object gas, and a current that flows due to the oxygen generated by the decomposition of the water vapor.

The pump voltage Vp0 applied to the oxygen pump cell 21 may be set as a value such that all or substantially all of water vapor in the measurement-object gas is decomposed in the internal cavity 25 (especially, around the inner oxygen pump electrode 22). The pump voltage Vp0 may vary depending on the intended use of the gas sensor 100, the configuration of the sensor element 101 and the like, and the pump voltage Vp0 may be, for example, about 800 mV or more and about 1500 mV or less, or, about 1000 mV or more and about 1500 mV or less.

When a pump voltage Vp1 is applied between the inner hydrogen pump electrode 32 and the outer hydrogen pump electrode 33 so that hydrogen is pumped out from the internal cavity 25 to the external space, a hydrogen pump current Ip1 increases as the pump voltage Vp1 is increased while the pump voltage Vp1 is low. Subsequently, when the pump voltage Vp1 becomes high, the hydrogen pump current Ip1 does not increase even when the pump voltage Vp1 is increased, and becomes to be saturated. A value of the saturated current at this time is referred to as a limiting current value. A region in which the hydrogen pump current Ip1 is at the limiting current value with respect to the pump voltage Vp1 is referred to as a limiting current region. In the limiting current region, it is considered that substantially all of hydrogen that reaches the inner hydrogen pump electrode 32 is pumped out by the hydrogen pump cell 31.

In driving the gas sensor 100, the pump control part 92 applies the predetermined pump voltage Vp1 between the intracavity hydrogen pump electrode (the inner hydrogen pump electrode 32) and the extracavity hydrogen pump electrode (the outer hydrogen pump electrode 33) in the hydrogen pump cell 31 by the variable power supply 34 to pump out hydrogen generated by the decomposition of the water vapor at the inner oxygen pump electrode 22 from the internal cavity 25. In this case, the hydrogen pump current Ip1 flowing through the hydrogen pump cell 31 flows from the outer hydrogen pump electrode 33 toward the inner hydrogen pump electrode 32 in the outside of the sensor element 101. In other words, the oxygen pump current Ip0 and the hydrogen pump current Ip1 are opposite in a current direction.

The pump voltage Vp1 applied to the hydrogen pump cell 31 may be set as a value such that all of, or substantially all of hydrogen generated by the decomposition of the water vapor at the inner oxygen pump electrode 22 is pumped out from the internal cavity 25 (especially, around the inner hydrogen pump electrode 32). The pump voltage Vp1 may vary depending on the intended use of the gas sensor 100, the configuration of the sensor element 101 and the like, and the pump voltage Vp1 may be, for example, about 100 mV or more and about 500 mV or less.

The concentration calculating part 93 is configured to calculate a concentration of a target gas to be measured (in this embodiment, each of O₂ concentration and H₂O concentration) in a measurement-object gas. The concentration calculating part 93 calculates the concentration of the target gas to be measured based on at least one of the oxygen pump current Ip0 and the hydrogen pump current Ip1.

The concentration calculating part 93 acquires the hydrogen pump current Ip1 in the hydrogen pump cell 31, calculates H₂O concentration in a measurement-object gas on the basis of a previously-stored conversion parameter (current-H₂O concentration conversion parameter) between the hydrogen pump current Ip1 and the H₂O concentration in the measurement-object gas, and outputs the H₂O concentration as a measurement value of the gas sensor 100. The current-H₂O concentration conversion parameter is previously stored in the memory of the control part 91 which functions as the concentration calculating part 93. The current-H₂O concentration conversion parameter may appropriately be determined by those skilled in the art by, for example, previously performing an experiment on the gas sensor 100. The current-H₂O concentration conversion parameter may be, for example, the coefficient of an approximate expression (e.g., linear function) obtained by experiment or a map showing the relationship between the hydrogen pump current Ip1 and the H₂O concentration in a measurement-object gas. The current-H₂O concentration conversion parameter may be specific to each individual gas sensor 100 or may be common to a plurality of gas sensors.

The concentration calculating part 93 acquires the oxygen pump current Ip0 in the oxygen pump cell 21 and the hydrogen pump current Ip1 in the hydrogen pump cell 31, calculates O₂ concentration in the measurement-object gas on the basis of a previously-stored conversion parameter (current-O₂ concentration conversion parameter) between these currents and the oxygen concentration (O₂ concentration) in the measurement-object gas, and outputs the O₂ concentration as a measurement value of the gas sensor 100. The current-O₂ concentration conversion parameter is previously stored, as data showing a relationship between the pump currents Ip0, Ip1 and the O₂ concentration in the measurement-object gas, in the memory of the control part 91 which functions as the concentration calculating part 93. The current-O₂ concentration conversion parameter may appropriately be determined by those skilled in the art by, for example, previously performing an experiment on the gas sensor 100. The current-O₂ concentration conversion parameter may be, for example, the coefficient of an approximate expression (e.g., linear function) obtained by experiment or a map showing the relationship between the pump currents Ip0, Ip1 and the O₂ concentration in a measurement-object gas. The current-O₂ concentration conversion parameter may be specific to each individual gas sensor 100 or may be common to a plurality of gas sensors.

The concentration calculating part 93 calculates the O₂ concentration and the H₂O concentration simultaneously, or, in parallel. Thus, the gas sensor 100 is so configured to be able to measure each concentration of a plurality of target gases to be measured (in this embodiment, two kinds, O₂ concentration and H₂O concentration). Alternatively, the gas sensor 100 may be configured to measure only one of the O₂ concentration and the H₂O concentration.

### (Measurement of Oxygen Concentration and Water Vapor Concentration in the Measurement-Object Gas)

Next, a method for measuring each concentration of oxygen O₂ and water vapor H₂O in the measurement-object gas by using the gas sensor 100 having such a configuration as described above will be described.

The measurement-object gas is introduced from the gas inlet 10, passes through the first diffusion-rate limiting path 11, the buffer space 12, and the fourth diffusion-rate limiting path 13 in this order so that a predetermined diffusion resistance is imparted to the measurement-object gas, and reaches the internal cavity 25.

When the pump control part 92 controls the operation of the oxygen pump cell 21 as described above, all or substantially all of water vapor in the measurement-object gas is decomposed (2H₂O→2H₂+O₂) in the internal cavity 25 (especially, in the vicinity of a surface of the inner oxygen pump electrode 22) to generate hydrogen and oxygen. All or substantially all of oxygen generated by the decomposition of the water vapor and oxygen originally existing in the measurement-object gas are pumped out from the internal cavity 25 by the oxygen pump cell 21.

The measurement-object gas, which contains hydrogen generated by the decomposition of the water vapor and does not substantially contain either water vapor or oxygen, reaches the inner hydrogen pump electrode 32 in the hydrogen pump cell 31.

When the pump control part 92 controls the operation of the hydrogen pump cell 31 as described above, all or substantially all of hydrogen is pumped out from the internal cavity 25. The hydrogen pump current Ip1 flowing through the hydrogen pump cell 31 is a current due to the hydrogen H₂ generated by the decomposition of the water vapor H₂O in the measurement-object gas. When substantially all of the water vapor H₂O in the measurement-object gas is decomposed at the inner oxygen pump electrode 22 of the oxygen pump cell 21, an amount of the hydrogen H₂ generated by the decomposition of the water vapor H₂O becomes an amount corresponding to an amount (or, a concentration) of the water vapor H₂O in the measurement-object gas. Accordingly, a current value of the hydrogen pump current Ip1 is considered to become a current value corresponding to the water vapor concentration in the measurement-object gas. It is considered that the current value of the hydrogen pump current Ip1 is substantially proportional to the water vapor concentration in the measurement-object gas. Such a relationship between the water vapor concentration in the measurement-object gas and the hydrogen pump current Ip1 may be determined in advance, and the concentration calculating part 93 may previously store this as the current-H₂O concentration conversion parameter. The concentration calculating part 93 can calculate the water vapor concentration in the measurement-object gas based on the hydrogen pump current Ip1 by using the current-H₂O concentration conversion parameter.

As described above, the oxygen pump current Ip0 flowing through the oxygen pump cell 21 includes the current due to the oxygen gas O₂ originally exiting in the measurement-object gas, and the current due to the oxygen O₂ generated by the decomposition of the water vapor H₂O in the measurement-object gas. On the other hand, the hydrogen pump current Ip1 flowing through the hydrogen pump cell 31 is the current due to the hydrogen H₂ generated by the decomposition of the water vapor H₂O in the measurement-object gas. Each of an amount of the oxygen O₂ generated by the decomposition of the water vapor H₂O and an amount of the hydrogen H₂ generated by the decomposition of the water vapor H₂O becomes an amount corresponding to an amount (or, a concentration) of the water vapor H₂O in the measurement-object gas, and it is therefore considered to be possible to acquire the current due to the oxygen O₂ generated by the decomposition of the water vapor H₂O in the oxygen pump current Ip0, from a current value of the hydrogen pump current Ip1 or a water vapor concentration calculated by the hydrogen pump current Ip1. Accordingly, oxygen concentration in the measurement-object gas can be calculated based on the oxygen pump current Ip0 and the hydrogen pump current Ip1. For example, a relationship between a concentration of oxygen pumped out by the oxygen pump cell 21 (namely, sum of oxygen in the measurement-object gas and oxygen generated by the decomposition of the water vapor) and the oxygen pump current Ip0, and a relationship between the water vapor concentration in the measurement-object gas and the hydrogen pump current Ip1 may be determined in advance, and the concentration calculating part 93 may previously store these as the current-O₂ concentration conversion parameter. The concentration calculating part 93 can calculate the oxygen concentration in the measurement-object gas based on the oxygen pump current Ip0 and the hydrogen pump current Ip1 by using the current-O₂ concentration conversion parameter.

As such, the gas sensor 100 can measure oxygen concentration and water vapor concentration in the measurement-object gas. The gas sensor 100 is capable of accurate measurement in both cases where oxygen and water vapor coexist in the measurement-object gas and where only one of oxygen and water vapor exists in the measurement-object gas. Such a gas sensor 100 is useful, for example, in measuring exhaust gas from a hydrogen engine.

As described above, in the sensor element 101, the insulator layer 62 is interposed between the second oxygen-ion conductor layer 6 and the proton conductor layer 7 (see FIG. 1 and FIG. 2). The second oxygen-ion conductor layer 6 and the proton conductor layer 7 do not directly contact with each other, and are electrically insulated from each other. Therefore, when the pump control part 92 operates the oxygen pump cell 21 and the hydrogen pump cell 31, no electrically interference occurs among electrode potentials and pump currents in the oxygen pump cell 21 and the hydrogen pump cell 31, and it is thus considered that oxygen concentration and water vapor concentration can be measured more accurately.

Further, when the sensor element 101 is formed by integral firing (co-firing) as will be described later, the presence of the insulator layer 62 prevents the second oxygen-ion conductor layer 6 and the proton conductor layer 7 from coming into direct contact with each other during firing. Thus, components in each of the layers do not mutually move between the layers during firing and it is more likely to obtain the second oxygen-ion conductor layer 6 and the proton conductor layer 7 with desired compositions, respectively, after firing. In other words, the insulator layer 62 functions as buffer layer that does not contain an alkaline earth metal so that mass transfer during co-firing can be suppressed. For example, a component such as an alkaline earth metal in the proton-conductive solid electrolyte that constitutes the proton conductor layer 7 does not move during firing and is not dissolved into the oxygen-ion-conductive solid electrolyte that constitutes the second oxygen-ion conductor layer 6. Therefore, the sensor element 101 can be manufactured so that the second oxygen-ion conductor layer 6 and the proton conductor layer 7 have the desired compositions, respectively. As a result, the sensor element 101 can exhibit desired oxygen ion conductivity and desired proton conductivity, and it is thus considered that oxygen concentration and water vapor concentration can be measured more accurately.

In the sensor element 101, as shown in FIG. 1 and FIG. 2, the extracavity oxygen pump electrode (the outer oxygen pump electrode 23) of the oxygen pump cell 21 and the extracavity hydrogen pump electrode (the outer hydrogen pump electrode 33) of the hydrogen pump cell 31 are both disposed at a position in contact with the measurement-object gas on the outer surface of the sensor element 101. The sensor element 101 have neither an oxygen reference electrode in contact with a reference gas (a gas with known oxygen concentration; e.g. air) that serves as a reference for oxygen concentration, nor a hydrogen reference electrode in contact with a reference gas (a gas with known hydrogen concentration) that serves as a reference for hydrogen concentration. Therefore, it is not necessary to form a space for introducing the reference gas inside the base part 102. As the internal structure of the base part 102 becomes more complex, for example, as the volume and/or the number of spaces present inside the base part 102 becomes larger, it is concerned that thermal stress may be generated in the sensor element 101 and a crack may be likely to occur in the internal structure of the sensor element 101. Since the internal structure of the base part 102 can be simplified in the sensor element 101, it is considered to be possible to keep thermal stress low during temperature rise or fall in the sensor element 101, and as a result, to suppress occurrence of the crack in the internal structure of the sensor element 101. “Temperature rise or fall in the sensor element 101” refers to, for example, a temperature rise due to heating when the gas sensor 100 starts to drive, a temperature drop due to cooling of the sensor element 101 by flow velocity and/or temperature of the measurement-object gas, or the like.

In the gas sensor of the present invention, as shown in FIG. 1 and FIG. 2, the intracavity hydrogen pump electrode (the inner hydrogen pump electrode 32) in the hydrogen pump cell 31 may be disposed at a position farther from the first diffusion-rate limiting path 11 than the intracavity oxygen pump electrode (the inner oxygen pump electrode 22) in the oxygen pump cell 21, that is, at a position rearward the intracavity oxygen pump electrode (the inner oxygen pump electrode 22). In this case, substantially all of water vapor in the measurement-object gas is decomposed at the inner oxygen pump electrode 22, oxygen generated by the decomposition of the water vapor is pumped out by the oxygen pump cell 21, and then, the measurement-object gas containing hydrogen generated by the decomposition of the water vapor reaches the inner hydrogen pump electrode 32. Therefore, the hydrogen generated by the decomposition of the water vapor can be pumped out by the hydrogen pump cell 31 more effectively.

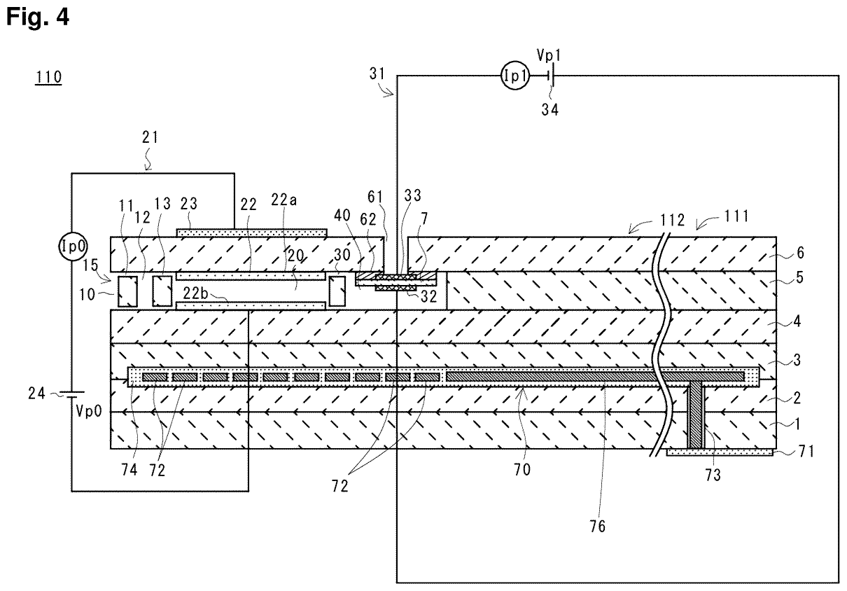

More preferably, an internal cavity in which the inner oxygen pump electrode 22 is disposed and an internal cavity in which the inner hydrogen pump electrode 32 is disposed may be separate internal cavities that communicate to each other via a diffusion-rate limiting path. That is, the sensor element 101 may have the structure having two internal cavities. FIG. 4 is a vertical sectional schematic view in the longitudinal direction of a sensor element 111, showing one example of a schematic configuration of a gas sensor 110. In FIG. 4, the same member as in FIG. 1 is denoted by the same sign.

In the sensor element 111, a measurement-object gas flow cavity 15 has a gas inlet 10 that opens on a surface of the base part 112, a first internal cavity 20 that communicates with the gas inlet 10 via the first diffusion-rate limiting path 11, on an inner surface of the first internal cavity 20 at least the second oxygen-ion conductor layer 6 being present, and a second internal cavity 40 that communicates with the first internal cavity 20 via a second diffusion-rate limiting path 30 (namely, a second diffusion-rate limiting part), on an inner surface of the second internal cavity 40 the second oxygen-ion conductor layer 6 and the proton conductor layer 7 being present. The first internal cavity 20 is formed to face the first oxygen-ion conductor layer 4 and the second oxygen-ion conductor layer 6, and the second internal cavity 40 is formed to face the first oxygen-ion conductor layer 4, the second oxygen-ion conductor layer 6, and the proton conductor layer 7.

The first internal cavity 20 and the second internal cavity 40 constitute internal spaces of the sensor element 111, as in the case of the internal cavity 25 in the above-described sensor element 101. Each of the internal spaces is provided in such a manner that a portion of the spacer layer 5 is hollowed out, and the top of each of the internal spaces is defined by the lower surface of the second oxygen-ion conductor layer 6, the bottom of each of the internal spaces is defined by the upper surface of the first oxygen-ion conductor layer 4, and the lateral surface of each of the internal spaces is defined by the lateral surface of the spacer layer 5.

The penetrating hole 61 is formed in the second oxygen-ion conductor layer 6 at a position where the second internal cavity 40 exists. The proton conductor layer 7 is located substantially parallel to the second oxygen-ion conductor layer 6, and completely covers the penetrating hole 61 and is formed on substantially the entire ceiling surface of the second internal cavity 40 on the side of the lower surface of the second oxygen-ion conductor layer 6, that is, on the side of the surface facing the second internal cavity 40.

The second diffusion-rate limiting path 30 is provided as two laterally elongated slits (having the longitudinal direction of the openings in the direction perpendicular to the figure in FIG. 4), as in the case of the first diffusion-rate limiting path 11. The second diffusion-rate limiting path 30 may be in such a form that a desired diffusion resistance is created, but the form is not limited to the slits.

The intracavity oxygen pump electrode (the inner oxygen pump electrode 22) is disposed in the first internal cavity 20, and the intracavity hydrogen pump electrode (the inner hydrogen pump electrode 32) is disposed in the second internal cavity 40. Thus, the first internal cavity 20 is provided as a space for adjusting the oxygen partial pressure in the measurement-object gas and decomposing water vapor in the measurement-object gas, and the second internal cavity 40 is provided as a space for pumping out hydrogen generated by the decomposition of the water vapor to measure water vapor concentration.

In the gas sensor 110 having the sensor element 111, when the pump control part 92 operates the oxygen pump cell 21, substantially all of water vapor in the measurement-object gas is decomposed in the first internal cavity 20, and all or substantially all of oxygen generated by the decomposition of the water vapor and oxygen originally existing in the measurement-object gas are pumped out from the first internal cavity 20 by the oxygen pump cell 21.

The measurement-object gas, which contains hydrogen generated by the decomposition of the water vapor and does not substantially contain either water vapor or oxygen, is introduced into the second internal cavity 40 through the second diffusion-rate limiting path 30, and reaches the inner hydrogen pump electrode 32. Hydrogen is a smaller molecule than water vapor and has a large enough diffusion coefficient so as to smoothly reach the inner hydrogen pump electrode 32 and be pumped out by the hydrogen pump cell 31. Therefore, it is considered that water vapor concentration can be detected more accurately.

### Embodiment 2

As a gas sensor 200 of Embodiment 2, one example of a gas sensor that detects oxygen O₂, water vapor H₂O, and carbon dioxide CO₂ in a measurement-object gas, and measures the concentrations of O₂, H₂O and CO₂ is shown. The gas sensor 200 is an example of a configuration provided with a sensor element 201 having two internal cavities.

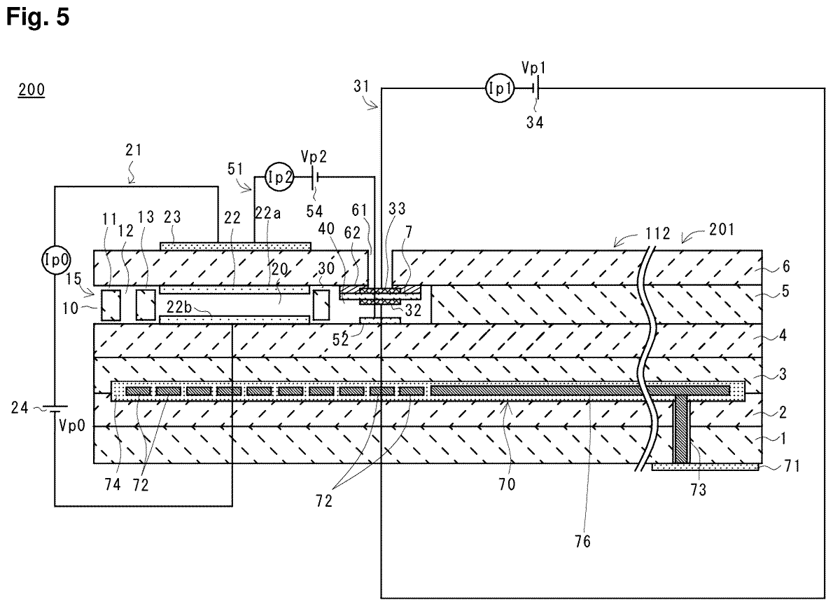

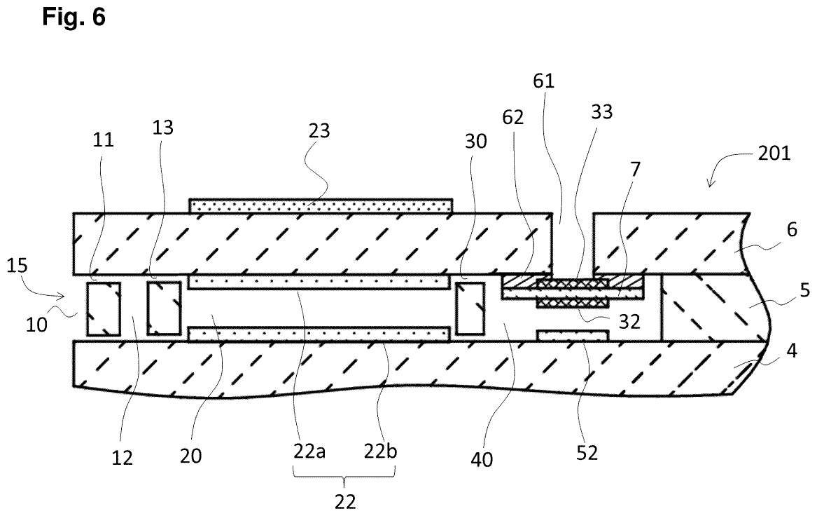

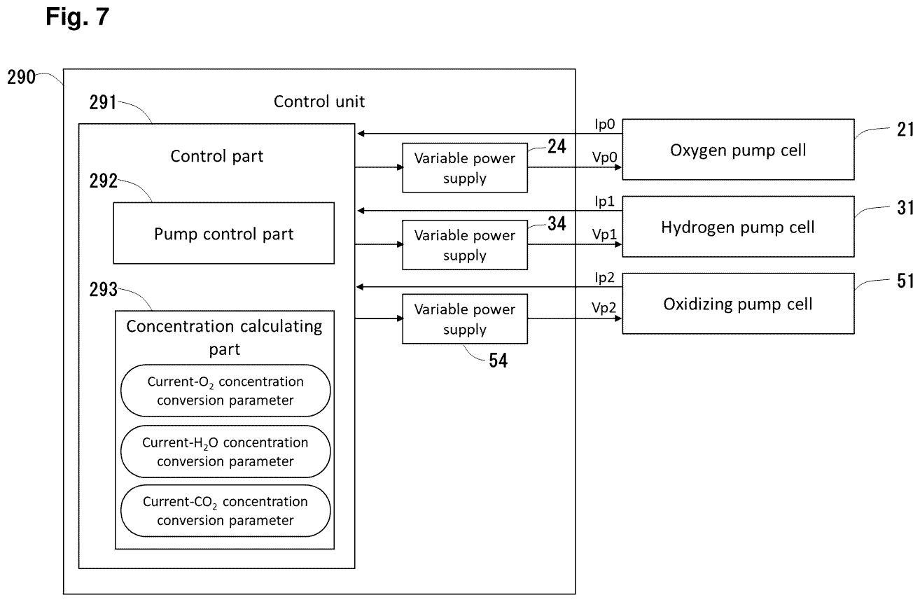

FIG. 5 is a vertical sectional schematic view in the longitudinal direction of the sensor element 201, showing one example of a schematic configuration of the gas sensor 200 of Embodiment 2. In FIG. 5, the same member as in FIG. 4 is denoted by the same sign. FIG. 6 is a partial sectional schematic view in the same section of FIG. 5, showing an arrangement of a measurement-object gas flow part 15 and its surrounding in the sensor element 201. FIG. 7 is a block diagram showing electric connections between a control unit 290 and the sensor element 201 in the gas sensor 200 of Embodiment 2.

### (Sensor Element)

The sensor element 201 further includes, in addition to the oxygen pump cell 21 and hydrogen pump cell 31,
an oxidizing pump cell 51 including: an intra-cavity oxidizing electrode (in the sensor element 201, an inner oxidizing electrode 52) disposed at a position farther from the first diffusion-rate limiting path 11 than the intracavity oxygen pump electrode (the inner oxygen pump electrode 22) on the oxygen-ion-conductive solid electrolyte layer (in the sensor element 201, the first oxygen-ion conductor layer 4) in the internal cavity of the measurement-object gas flow cavity 15; and an extra-cavity oxidizing electrode disposed at a position different from the measurement-object gas flow cavity 15 on the base part 112 and adjacent to the intracavity oxidizing electrode via the oxygen-ion-conductive solid electrolyte layer (the first oxygen-ion conductor layer 4, the spacer layer 5, and the second oxygen-ion conductor layer 6). In the sensor element 201, the inner oxidizing electrode 52 is disposed in the second internal cavity 40 on the rear end side of (namely, at a position farther from the first diffusion-rate limiting path 11 than) the first internal cavity 20 where the inner oxygen pump electrode 22 is disposed. Further, in the sensor element 200, the outer oxygen pump electrode 23 functions also as the extracavity oxidizing electrode.

That is, the oxidizing pump cell 51 is an electrochemical pump cell composed of the inner oxidizing electrode 52 disposed on the upper surface of the first oxygen-ion conductor layer 4 that defines the bottom surface of the second internal cavity 40, the outer oxygen pump electrode 23 disposed so as to be exposed to the external space, and the first oxygen-ion conductor layer 4, the spacer layer 5, and the second oxygen-ion conductor layer 6 sandwiched between the inner oxidizing electrode 52 and the outer oxygen pump electrode 23.

The inner oxidizing electrode 52 is a porous cermet electrode (an electrode in a state that a metal component and a ceramic component are mixed), as in the case of the inner oxygen pump electrode 22. The ceramic component to be used is not particularly limited, but is preferably an oxygen-ion-conductive solid electrolyte as in the case of the second oxygen-ion conductor layer 6 and the first oxygen-ion conductor layer 4. For example, ZrO₂ (stabilized ZrO₂) can be used as the ceramic component.

The inner oxidizing electrode 52 preferably contain a noble metal having catalytic activity (e.g., at least one of Pt, Rh, Ir, Ru, and Pd) as the metal component, as in the case of the inner oxygen pump electrode 22. For example, the inner oxidizing electrode 52 may be a porous cermet electrode made of Pt and ZrO₂.

In the oxidizing pump cell 51, a desired pump voltage Vp2 is applied between the inner oxidizing electrode 52 and the outer oxygen pump electrode 23 by a variable power supply 54 to make an oxidizing pump current Ip2 flow between the inner oxidizing electrode 52 and the outer oxygen pump electrode 23, and thus it is possible to pump oxygen into the second internal cavity 40 from the external space.

### (Control Unit)

FIG. 7 is a block diagram showing electric connections between the control unit 290 and the respective pump cells 21, 31 and 51 of the sensor element 201 in the gas sensor 200 of Embodiment 2. The control unit 290 includes the variable power supplies 24, 34 and 54 and a control part 291. The control part 291 includes a pump control part 292 and a concentration calculating part 293.

The control part 291 is configured to acquire a pump current (Ip0, Ip1, Ip2) in each of the pump cells 21, 31 and 51 of the sensor element 201. Further, the control part 291 is configured to output control signals to the variable power supplies 24, 34 and 54.

In the gas sensor 200, the pump control part 292 is configured to control the operation of the oxygen pump cell 21, the hydrogen pump cell 31 and the oxidizing pump cell 51 so as to measure a concentration of a target gas to be measured (in this embodiment, each of oxygen O₂, water vapor H₂O, and carbon dioxide CO₂) in a measurement-object gas.

The pump control part 292 applies a predetermined pump voltage Vp0 between the intracavity oxygen pump electrode (the inner oxygen pump electrode 22) and the extracavity oxygen pump electrode (the outer oxygen pump electrode 23) in the oxygen pump cell 21 to make an oxygen pump current Ip0 flow through the oxygen pump cell 21; applies a predetermined pump voltage Vp1 between the intracavity hydrogen pump electrode (the inner hydrogen pump electrode 32) and the extracavity hydrogen pump electrode (the outer hydrogen pump electrode 33) in the hydrogen pump cell 31 to make a hydrogen pump current Ip1 flow through the hydrogen pump cell 31; and applies a predetermined pump voltage Vp2 between the intracavity oxidizing electrode (the inner oxidizing electrode 52) and the extracavity oxidizing electrode (the outer oxygen pump electrode 23) in the oxidizing pump cell 51 to make an oxidizing pump current Ip2 flow through the oxidizing pump cell 51.

The pump control part 292 applies the predetermined pump voltage Vp0 between the intracavity oxygen pump electrode (the inner oxygen pump electrode 22) and the extracavity oxygen pump electrode (the outer oxygen pump electrode 23) by the variable power supply 24 to pump out oxygen in the measurement-object gas from the first internal cavity 20. At this time, water vapor and carbon dioxide in the measurement-object gas is respectively decomposed (or, reduced) at the inner oxygen pump electrode 22, and all of oxygen generated by the decomposition of the water vapor, oxygen generated by the decomposition of the carbon dioxide and oxygen originally existing in the measurement-object gas are pumped out from the first internal cavity 20. In this case, the oxygen pump current Ip0 flowing through the oxygen pump cell 21 flows from the inner oxygen pump electrode 22 toward the outer oxygen pump electrode 23 in the outside of the sensor element 201. The oxygen pump current Ip0 includes a current that flows due to the oxygen gas originally exiting in the measurement-object gas, a current that flows due to the oxygen generated by the decomposition of the water vapor, and a current that flows due to the oxygen generated by the decomposition of the carbon dioxide. It is to be noted that hydrogen generated by the decomposition of the water vapor and carbon monoxide generated by the decomposition of the carbon dioxide is guided to the second internal cavity 40 though the second diffusion-rate limiting path 30

The pump voltage Vp0 applied to the oxygen pump cell 21 may be set as a value such that all or substantially all of water vapor and carbon dioxide in the measurement-object gas is decomposed in the first internal cavity 20 (especially, around the inner oxygen pump electrode 22). The pump voltage Vp0 may vary depending on the intended use of the gas sensor 200, the configuration of the sensor element 201 and the like, and the pump voltage Vp0 may be, for example, about 800 mV or more and about 1500 mV or less, or, about 1000 mV or more and about 1500 mV or less.

The pump control part 292 applies, as in the case of the pump control part 92, the predetermined pump voltage Vp1 between the intracavity hydrogen pump electrode (the inner hydrogen pump electrode 32) and the extracavity hydrogen pump electrode (the outer hydrogen pump electrode 33) in the hydrogen pump cell 31 by the variable power supply 34 to pump out hydrogen generated by the decomposition of the water vapor in the first internal cavity 20 from the second internal cavity 40. In this case, the hydrogen pump current Ip1 flowing through the hydrogen pump cell 31 flows from the outer hydrogen pump electrode 33 toward the inner hydrogen pump electrode 32 in the outside of the sensor element 201. In other words, the oxygen pump current Ip0 and the hydrogen pump current Ip1 are opposite in a current direction.

The pump voltage Vp1 applied to the hydrogen pump cell 31 may be set as a value such that all or substantially all of hydrogen in the measurement-object gas introduced into the second internal cavity 40 is pumped out from the second internal cavity 40 (especially, around the inner hydrogen pump electrode 32). The pump voltage Vp1 may vary depending on the intended use of the gas sensor 200, the configuration of the sensor element 201 and the like, and the pump voltage Vp1 may be, for example, about 100 mV or more and about 500 mV or less.

Further, the pump control part 292 applies the predetermined pump voltage Vp2 between the intracavity oxidizing electrode (the inner oxidizing electrode 52) and the extracavity oxidizing electrode (the outer oxygen pump electrode 23) in the oxidizing pump cell 51 by the variable power supply 54 to pump oxygen into the second internal cavity 40 and oxidize (or, burn) carbon monoxide generated by the decomposition of the carbon dioxide in the first internal cavity 20. In this case, the oxidizing pump current Ip2 flowing through the oxidizing pump cell 51 flows from the outer oxygen pump electrode 23 toward the inner oxidizing electrode 52 in the outside of the sensor element 201. In other words, the oxygen pump current Ip0 and the oxidizing pump current Ip2 are opposite in a current direction. Referring to FIG. 5, in the gas sensor 200 of the present embodiment, the oxygen pump current Ip0 is detected as a positive current, and the oxidizing pump current Ip2 is detected as a negative current.

The pump voltage Vp2 applied to the oxidizing pump cell 51 may be set as a value such that all or substantially all of carbon monoxide in the measurement-object gas introduced into the second internal cavity 40 is decomposed in the second internal cavity 40 (especially, around the inner oxidizing electrode 52). The pump voltage Vp2 may vary depending on the intended use of the gas sensor 200, the configuration of the sensor element 201 and the like, and the pump voltage Vp2 may be, for example, about 100 mV or more and about 200 mV or less.

The concentration calculating part 293 is configured to calculate a concentration of a target gas to be measured (in this embodiment, each of O₂ concentration, H₂O concentration, and CO₂ concentration) in a measurement-object gas. The concentration calculating part 293 calculates the concentration of the target gas to be measured based on at least one of the oxygen pump current Ip0, the hydrogen pump current Ip1, and the oxidizing pump current Ip2.

The concentration calculating part 293, as in the case of the concentration calculating part 93, acquires the hydrogen pump current Ip1 in the hydrogen pump cell 31, calculates H₂O concentration in a measurement-object gas on the basis of a previously-stored conversion parameter (current-H₂O concentration conversion parameter) between the hydrogen pump current Ip1 and the H₂O concentration in the measurement-object gas, and outputs the H₂O concentration as a measurement value of the gas sensor 200. The current-H₂O concentration conversion parameter is previously stored in the memory of the control part 291 which functions as the concentration calculating part 293. The current-H₂O concentration conversion parameter may appropriately be determined by those skilled in the art by, for example, previously performing an experiment on the gas sensor 200. The current-H₂O concentration conversion parameter may be, for example, the coefficient of an approximate expression (e.g., linear function) obtained by experiment or a map showing the relationship between the hydrogen pump current Ip1 and the H₂O concentration in a measurement-object gas. The current-H₂O concentration conversion parameter may be specific to each individual gas sensor 200 or may be common to a plurality of gas sensors.

The concentration calculating part 293 acquires the oxidizing pump current Ip2 in the oxidizing pump cell 51, calculates CO₂ concentration in a measurement-object gas on the basis of a previously-stored conversion parameter (current-CO₂ concentration conversion parameter) between the oxidizing pump current Ip2 and the CO₂ concentration in the measurement-object gas, and outputs the CO₂ concentration as a measurement value of the gas sensor 200. The current-CO₂ concentration conversion parameter is previously stored in the memory of the control part 291 which functions as the concentration calculating part 293. The current-CO₂ concentration conversion parameter may appropriately be determined by those skilled in the art by, for example, previously performing an experiment on the gas sensor 200. The current-CO₂ concentration conversion parameter may be, for example, the coefficient of an approximate expression (e.g., linear function) obtained by experiment or a map showing the relationship between the oxidizing pump current Ip2 and the CO₂ concentration in a measurement-object gas. The current-CO₂ concentration conversion parameter may be specific to each individual gas sensor 200 or may be common to a plurality of gas sensors.

The concentration calculating part 293 acquires the oxygen pump current Ip0 in the oxygen pump cell 21, the hydrogen pump current Ip1 in the hydrogen pump cell 31 and the oxidizing pump current Ip2 in the oxidizing pump cell 51, calculates O₂ concentration in the measurement-object gas on the basis of a previously-stored conversion parameter (current-O₂ concentration conversion parameter) between these currents and the oxygen concentration (O₂ concentration) in the measurement-object gas, and outputs the O₂ concentration as a measurement value of the gas sensor 200. The current-O₂ concentration conversion parameter is previously stored, as data showing a relationship between the pump currents Ip0, Ip1, Ip2 and the O₂ concentration in the measurement-object gas, in the memory of the control part 291 which functions as the concentration calculating part 293. The current-O₂ concentration conversion parameter may appropriately be determined by those skilled in the art by, for example, previously performing an experiment on the gas sensor 200. The current-O₂ concentration conversion parameter may be, for example, the coefficient of an approximate expression (e.g., linear function) obtained by experiment or a map showing the relationship between the pump currents Ip0, Ip1, Ip2 and the O₂ concentration in a measurement-object gas. The current-O₂ concentration conversion parameter may be specific to each individual gas sensor 200 or may be common to a plurality of gas sensors.

The concentration calculating part 293 calculates the O₂ concentration, the H₂O concentration, and the CO₂ concentration simultaneously, or, in parallel. Thus, the gas sensor 200 is so configured to measure each concentration of a plurality of target gases to be measured (in this embodiment, three kinds, O₂ concentration, H₂O concentration, and CO₂ concentration). Alternatively, the gas sensor 200 may be configured to measure only one or two of the O₂ concentration, the H₂O concentration, and the CO₂ concentration.

### (Measurement of Oxygen Concentration, Water Vapor Concentration, and Carbon Dioxide Concentration in the Measurement-Object Gas)

Next, a method for measuring each concentration of oxygen O₂, water vapor H₂O, and carbon dioxide CO₂ in the measurement-object gas by using the gas sensor 200 having such a configuration as described above will be described.

The measurement-object gas is introduced from the gas inlet 10, passes through the first diffusion-rate limiting path 11, the buffer space 12, and the fourth diffusion-rate limiting path 13 in this order so that a predetermined diffusion resistance is imparted to the measurement-object gas, and reaches the first internal cavity 20.

When the pump control part 292 controls the operation of the oxygen pump cell 21 as described above, all or substantially all of water vapor in the measurement-object gas is decomposed (2H₂O→2H₂+O₂) in the first internal cavity 20 (especially, in the vicinity of a surface of the inner oxygen pump electrode 22) to generate hydrogen and oxygen. Also, all or substantially all of carbon dioxide in the measurement-object gas is decomposed (2CO₂→2CO+O₂) to generate carbon monoxide and oxygen. All or substantially all of oxygen generated by the decomposition of the water vapor, oxygen generated by the decomposition of the carbon dioxide, and oxygen originally existing in the measurement-object gas are pumped out from the first internal cavity 20 by the oxygen pump cell 21.

The measurement-object gas, which contains hydrogen generated by the decomposition of the water vapor and carbon monoxide generated by the decomposition of the carbon dioxide, and does not substantially contain either water vapor, carbon dioxide, or oxygen, passes through the second diffusion-rate limiting path 30 so that a predetermined diffusion resistance is imparted to the measurement-object gas, and reaches the second internal cavity 40.

When the pump control part 292 controls the operation of the hydrogen pump cell 31 as described above, all or substantially all of hydrogen is pumped out from the second internal cavity 40. The hydrogen pump current Ip1 flowing through the hydrogen pump cell 31 is a current due to the hydrogen H₂ generated by the decomposition of the water vapor H₂O in the measurement-object gas. When substantially all of the water vapor H₂O in the measurement-object gas is decomposed at the inner oxygen pump electrode 22 of the oxygen pump cell 21, an amount of the hydrogen H₂ generated by the decomposition of the water vapor H₂O becomes an amount corresponding to an amount (or, a concentration) of the water vapor H₂O in the measurement-object gas. Accordingly, a current value of the hydrogen pump current Ip1 is considered to become a current value corresponding to the water vapor concentration in the measurement-object gas. It is considered that the current value of the hydrogen pump current Ip1 is substantially proportional to the water vapor concentration in the measurement-object gas. Such a relationship between the water vapor concentration in the measurement-object gas and the hydrogen pump current Ip1 may be determined in advance, and the concentration calculating part 293 may previously store this as the current-H₂O concentration conversion parameter. The concentration calculating part 293 can calculate the water vapor concentration in the measurement-object gas based on the hydrogen pump current Ip1 by using the current-H₂O concentration conversion parameter.

When the pump control part 292 controls the operation of the oxidizing pump cell 51 as described above, oxygen is pumped into the second internal cavity 40 so that all or substantially all of carbon monoxide that reaches the second internal cavity 40 is oxidized. The oxidizing pump current Ip2 flowing through the oxidizing pump cell 51 is a current due to the carbon monoxide CO generated by the decomposition of the carbon dioxide CO₂. When substantially all of the carbon dioxide CO₂ in the measurement-object gas is decomposed at the inner oxygen pump electrode 22 of the oxygen pump cell 21, an amount of the carbon monoxide CO generated by the decomposition of the carbon dioxide CO₂ becomes an amount corresponding to an amount (or, a concentration) of the carbon dioxide CO₂ in the measurement-object gas. Therefore, an amount of oxygen pumped-in by the oxidizing pump cell 51 to oxidize the carbon monoxide CO becomes the amount corresponding to the amount (or, the concentration) of the carbon dioxide CO₂ in the measurement-object gas. Accordingly, a current value of the oxidizing pump current Ip2 is considered to become a current value corresponding to the carbon dioxide concentration in the measurement-object gas. Such a relationship between the carbon dioxide concentration in the measurement-object gas and the oxidizing pump current Ip2 may be determined in advance, and the concentration calculating part 293 may previously store this as the current-CO₂ concentration conversion parameter. The concentration calculating part 293 can calculate the carbon dioxide concentration in the measurement-object gas based on the oxidizing pump current Ip2 by using the current-CO₂ concentration conversion parameter.

As described above, the oxygen pump current Ip0 flowing through the oxygen pump cell 21 includes the current due to the oxygen gas O₂ originally exiting in the measurement-object gas, the current due to the oxygen O₂ generated by the decomposition of the water vapor H₂O in the measurement-object gas, and the current due to the oxygen O₂ generated by the decomposition of the carbon dioxide CO₂ in the measurement-object gas. On the other hand, the hydrogen pump current Ip1 flowing through the hydrogen pump cell 31 is the current due to the hydrogen H₂ generated by the decomposition of the water vapor H₂O in the measurement-object gas. Further, the oxidizing pump current Ip2 flowing through the oxidizing pump cell 51 is the current due to the carbon monoxide CO generated by the decomposition of the carbon dioxide CO₂ in the measurement-object gas. Each of an amount of the oxygen O₂ generated by the decomposition of the water vapor H₂O and an amount of the hydrogen H₂ generated by the decomposition of the water vapor H₂O becomes an amount corresponding to an amount (or, a concentration) of the water vapor H₂O in the measurement-object gas, and it is therefore considered to be possible to acquire the current due to the oxygen O₂ generated by the decomposition of the water vapor H₂O in the oxygen pump current Ip0, from a current value of the hydrogen pump current Ip1 or a water vapor concentration calculated by the hydrogen pump current Ip1. Each of an amount of the oxygen O₂ generated by the decomposition of the carbon dioxide CO₂ and an amount of the carbon monoxide CO generated by the decomposition of the carbon dioxide CO₂ becomes an amount corresponding to an amount (or, a concentration) of the carbon dioxide CO₂ in the measurement-object gas, and it is therefore considered to be possible to acquire the current due to the oxygen O₂ generated by the decomposition of the carbon dioxide CO₂ in the oxygen pump current Ip0, from a current value of the oxidizing pump current Ip2 or a carbon dioxide concentration calculated by the oxidizing pump current Ip2. Accordingly, oxygen concentration in the measurement-object gas can be calculated based on the oxygen pump current Ip0, the hydrogen pump current Ip1 and the oxidizing pump current Ip2. For example, a relationship between a concentration of oxygen pumped out by the oxygen pump cell 21 (namely, sum of oxygen in the measurement-object gas, oxygen generated by the decomposition of the water vapor, and oxygen generated by the decomposition of the carbon dioxide) and the oxygen pump current Ip0, a relationship between the water vapor concentration in the measurement-object gas and the hydrogen pump current Ip1, and a relationship between the carbon dioxide concentration in the measurement-object gas and the oxidizing pump current Ip2 may be determined in advance, and the concentration calculating part 293 may previously store these as the current-O₂ concentration conversion parameter. The concentration calculating part 293 can calculate the oxygen concentration in the measurement-object gas based on the oxygen pump current Ip0, the hydrogen pump current Ip1 and the oxidizing pump current Ip2 by using the current-O₂ concentration conversion parameter.

As such, the gas sensor 200 can measure oxygen concentration, water vapor concentration and carbon dioxide concentration in the measurement-object gas. The gas sensor 200 is capable of accurate measurement in both cases where oxygen, water vapor and carbon dioxide coexist in the measurement-object gas and where any one or two of oxygen, water vapor and carbon dioxide exist in the measurement-object gas. Such a gas sensor 200 is useful, for example, in measuring exhaust gas from a gasoline engine or a diesel engine.

In the sensor element 201, the outer oxygen pump electrode 23 has two functions as an extracavity oxygen pump electrode in the oxygen pump cell 21 and an extracavity oxidizing electrode in the oxidizing pump cell 51. However, the extracavity oxidizing electrode may be formed as an electrode different from the outer oxygen pump electrode 23.

In the sensor element 201, the intracavity oxidizing electrode (the inner oxidizing electrode 52) is disposed on the first oxygen-ion conductor layer 4 of the bottom surface of the second internal cavity 40. However, the intracavity oxidizing electrode is not limited thereto. The intracavity oxidizing electrode (the inner oxidizing electrode 52) may be disposed at a position farther from the first diffusion-rate limiting path 11 than the intracavity oxygen pump electrode (the inner oxygen pump electrode 22) in the oxygen pump cell 21, that is, at a position rearward the intracavity oxygen pump electrode (the inner oxygen pump electrode 22). For example, a region where the second oxygen-ion conductor layer 6 is present may be provided on the ceiling surface of the second internal cavity 40, and the intracavity hydrogen pump electrode (the inner hydrogen pump electrode 32) and the intracavity oxidizing electrode (the inner oxidizing electrode 52) may be arranged in parallel or in series on the ceiling surface of the second internal cavity 40.

More preferably, the intracavity oxidizing electrode (the inner oxidizing electrode 52) may be disposed on the oxygen-ion-conductive solid electrolyte layer at a position farther from the first diffusion-rate limiting path 11 than the intracavity hydrogen pump electrode (the inner hydrogen pump electrode 32). In this case, the measurement-object gas which contains hydrogen generated by the decomposition of the water vapor and carbon monoxide generated by the decomposition of the carbon dioxide reaches the inner hydrogen pump electrode 32, and substantially all of the hydrogen is pumped out by the hydrogen pump cell 31. Then, the measurement-object gas which contains the carbon monoxide generated by the decomposition of the carbon dioxide reaches the inner oxidizing electrode 52. Therefore, the carbon monoxide may be oxidized more precisely in the oxidizing pump cell 51. As a result, it is considered that carbon dioxide concentration can be measured more accurately.

The intracavity oxidizing electrode (the inner oxidizing electrode 52) may be disposed, for example, on the first oxygen-ion conductor layer 4 or the second oxygen-ion conductor layer 6 in the second internal cavity 40 at a position farther from the second diffusion-rate limiting path 30 in a longitudinal direction of the sensor element 201 (the base part 112) than the intracavity hydrogen pump electrode (the inner hydrogen pump electrode 32).

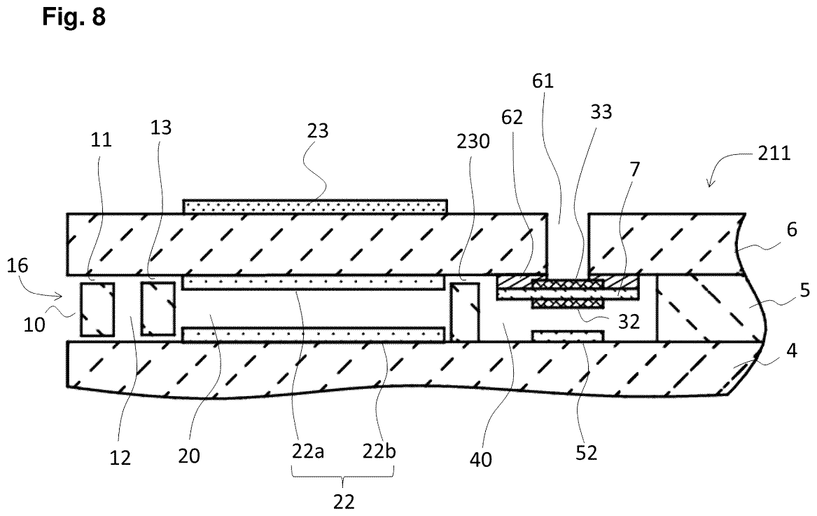

For example, as shown in FIG. 8, the second diffusion-rate limiting path may be provided only between the second oxygen-ion conductor layer 6 and the spacer layer 5 to make the inner oxidizing electrode 52 exist at a position farther from the first diffusion-rate limiting path 11 than the inner hydrogen pump electrode 32. FIG. 8 is a partial sectional schematic view in the same section of FIG. 5, showing a sensor element 211 as another example of arrangement of a measurement-object gas flow part and its surrounding. In a measurement-object gas flow part 16 of the sensor element 211, a second diffusion-rate limiting path 230 is provided as a single laterally elongated slit (having the longitudinal direction of the opening in the direction perpendicular to the figure in FIG. 8) between the second oxygen-ion conductor layer 6 and the spacer layer 5. Therefore, a gas diffusion distance between the second diffusion-rate limiting path 230 and the inner oxidizing electrode 52 is longer than a gas diffusion distance between the second diffusion-rate limiting path 230 and the inner hydrogen pump electrode 32. Further, for example, a porous protective layer covering the inner oxidizing electrode 52 may be formed as a diffusion-rate limiting part between the inner hydrogen pump electrode 32 and the inner oxidizing electrode 52.

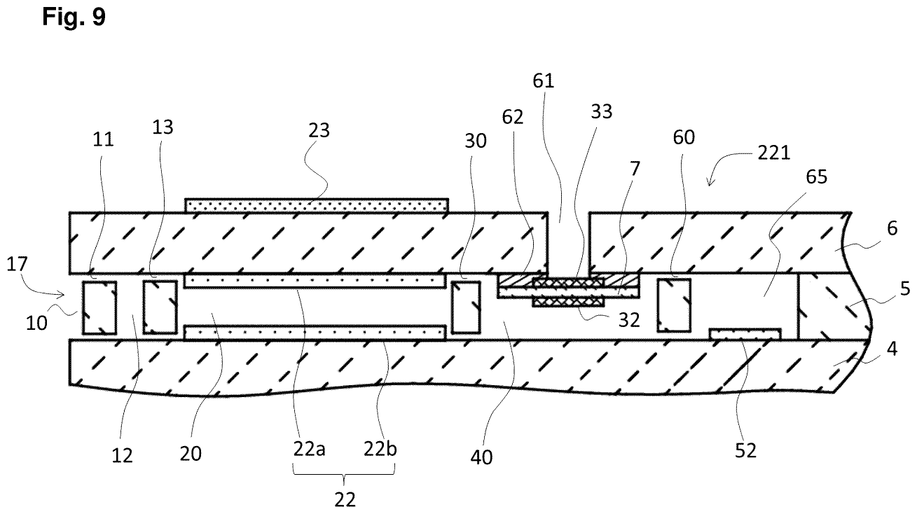

For example, as shown in FIG. 9, the sensor element may further have a third internal cavity 65, and the inner oxidizing electrode 52 may be disposed in the third internal cavity 65. FIG. 9 is a partial sectional schematic view in the same section of FIG. 5, showing a sensor element 221 as another example of arrangement of a measurement-object gas flow part and its surrounding. In the sensor element 221, a measurement-object gas flow cavity 17 has a gas inlet 10 that opens on a surface of the sensor element 221; a first internal cavity 20 that communicates with the gas inlet 10 via a first diffusion-rate limiting path 11, on an inner surface of the first internal cavity 20 at least the second oxygen-ion conductor layer 6 being present; a second internal cavity 40 that communicates with the first internal cavity 20 via a second diffusion-rate limiting path 30, on an inner surface of the second internal cavity 40 at least the proton conductor layer 7 being present, and the third internal cavity 65 that communicates with the second internal cavity 40 via a third diffusion-rate limiting path 60, on an inner surface of the third internal cavity 65 at least the second oxygen-ion conductor layer 6 being present. That is, the sensor element 221 shows an example of the structure having three internal cavities.

In the sensor element 221, the inner oxygen pump electrode 22 is disposed in the first internal cavity 20, the inner hydrogen pump electrode 32 is disposed in the second internal cavity 40, and the inner oxidizing electrode 52 is disposed in the third internal cavity 65. In the first internal cavity 20, decomposition of water vapor and carbon dioxide and pumping out of oxygen are performed; in the second internal cavity 40, pumping out of hydrogen generated by the decomposition of the water vapor is performed; and in the third internal cavity 65, oxidation of carbon monoxide generated by the decomposition of the carbon dioxide is performed. By dividing roles of respective internal cavities, it is considered that oxygen concentration, water vapor concentration and carbon dioxide concentration can be measured much more accurately.

### Embodiment 3

In the present invention, a sensor element may include a reference electrode disposed inside the base part to be in contact with a reference gas.

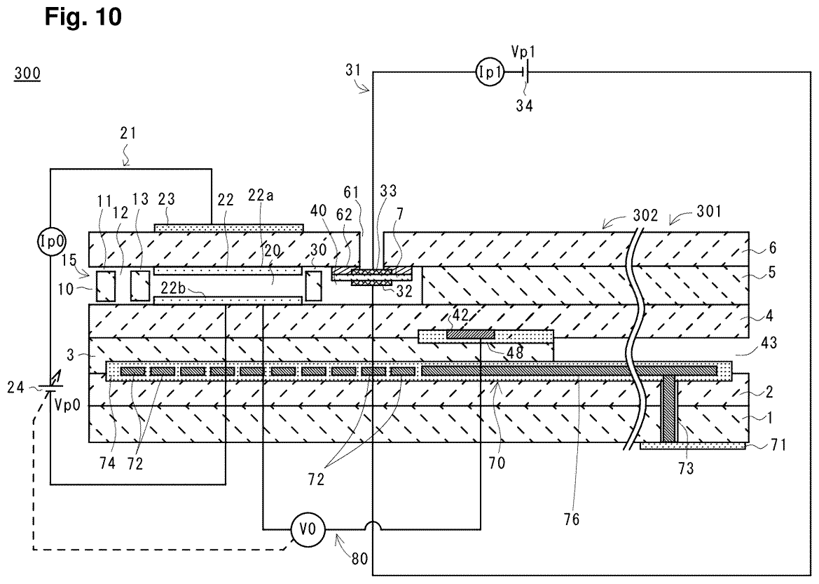

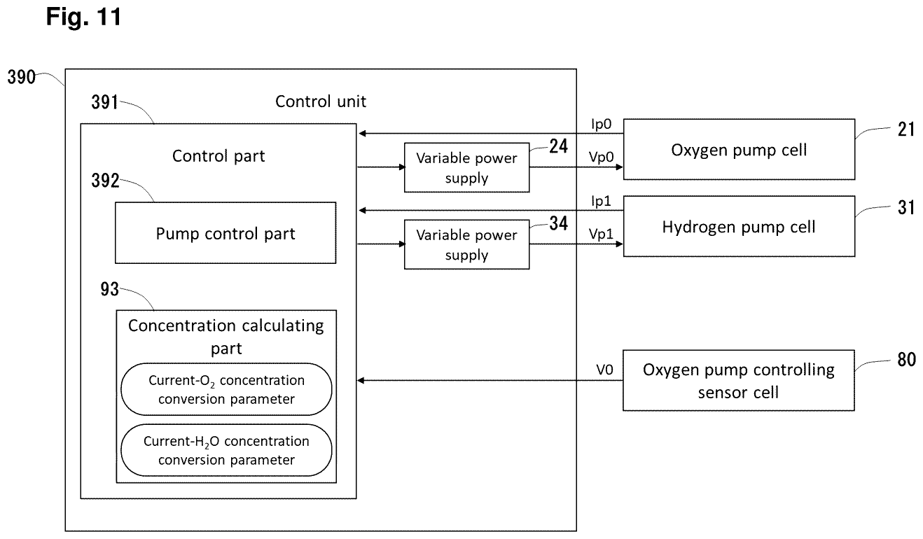

As a gas sensor 300 of Embodiment 3, an example including a sensor element 301 having a reference electrode 42 is shown. The gas sensor 300 of Embodiment 3 is an example of a gas sensor that detects oxygen O₂, and water vapor H₂O in a measurement-object gas, and measures the concentrations of O₂, and H₂O. The gas sensor 300 is an example of a configuration provided with the sensor element 301 having two internal cavities. FIG. 10 is a vertical sectional schematic view in the longitudinal direction of the sensor element 301, showing one example of a schematic configuration of the gas sensor 300 of Embodiment 3. In FIG. 10, the same member as in FIG. 4 is denoted by the same sign. FIG. 11 is a block diagram showing electric connections between the control unit 390 and the sensor element 301 in the gas sensor 300 of Embodiment 3.

In the sensor element 301, the arrangement of the measurement-object gas flow cavity 15 is the same as in the case of the sensor element 111 in the above-described gas sensor 110. In the sensor element 301, at a position farther from the front end than the measurement-object gas flow part 15, a reference gas introduction space 43 is disposed between the upper surface of the second substrate layer 2 and the lower surface of the first oxygen-ion conductor layer 4 at a position where the reference gas introduction space 43 is laterally defined by the lateral surface of the third substrate layer 3. The reference gas introduction space 43 has an opening in the other end part (hereinafter, referred to as a rear end part) of the sensor element 301. As a reference gas for oxygen concentration measurement, for example, air is introduced into the reference gas introduction space 43. In a width direction of the sensor element 301, the heater lead 76 may be disposed at a position where the reference gas introduction space 43 exists as shown in FIG. 10, or may be disposed so as to be sandwiched between the third substrate layer 3 and the second substrate layer 2 at a position where the reference gas introduction space 43 does not exist.

An air introduction layer 48 is a layer formed of porous alumina, and is so configured that a reference gas is introduced into the air introduction layer 48 via the reference gas introduction space 43. The air introduction layer 48 is formed to cover the reference electrode 42.

The reference electrode 42 is an electrode disposed inside the base part 302 to be in contact with a reference gas. Specifically, in the sensor element 301, the reference electrode 42 is an electrode sandwiched between the third substrate layer 3 and the lower surface of the first solid electrolyte layer 4, and as described above, the air introduction layer 48 leading to the reference gas introduction space 43 is disposed around the reference electrode 42. That is, the reference electrode 42 is disposed to be in contact with a reference gas via the air introduction layer 48 which is a porous material, and the reference gas introduction space 43. The reference electrode 42 is formed as a porous cermet electrode (e.g., a cermet electrode of Pt and ZrO₂) in a rectangular shape in a planar view.

In the sensor element 301, the reference electrode 42 electrochemically corresponds to the intracavity oxygen pump electrode (the inner oxygen pump electrode 22). That is, the reference electrode 42 is disposed adjacent to the intracavity oxygen pump electrode (the inner oxygen pump electrode 22) via the oxygen-ion-conductive solid electrolyte layer.

Specifically, the inner oxygen pump electrode 22, the second oxygen-ion conductor layer 6, the spacer layer 5, the first oxygen-ion conductor layer 4, and the reference electrode 42 form an electrochemical sensor cell, namely, an oxygen pump controlling sensor cell 80. The oxygen concentration (oxygen partial pressure) in the first internal cavity 20 can be detected from an electromotive force V0 measured between the inner oxygen pump electrode 22 and the reference electrode 42 in the oxygen pump controlling sensor cell 80.

In addition, a control is performed to apply the pump voltage Vp0 in the oxygen pump cell 21 based on the electromotive force V0. More specifically, the oxygen pump current Ip0 is controlled by performing feedback control of the pump voltage Vp0 so that the electromotive force V0 is constant. Thus, the oxygen concentration in the first internal cavity 20 can be controlled at a predetermined concentration, and it is therefore possible to more surely decompose substantially all of water vapor in the measurement-object gas and pump out oxygen in the first internal cavity 20. Therefore, water vapor concentration and oxygen concentration in the measurement-object gas can be detected with higher accuracy.

FIG. 11 is a block diagram showing electric connections between the control unit 390 and the respective pump cells 21 and 31 and the oxygen pump controlling sensor cell 80 of the sensor element 301 in the gas sensor 300 of Embodiment 3. In FIG. 11, the same member as in FIG. 3 is denoted by the same sign. The control unit 390 includes the variable power supplies 24 and 34 and a control part 391. The control part 391 includes a pump control part 392 and the concentration calculating part 93.

The control part 391 is configured to acquire a pump current (Ip0, Ip1) in each of the pump cells 21 and 31, and the electromotive force V0 in the oxygen pump controlling sensor cell 80 of the sensor element 301. Further, the control part 391 is configured to output control signals to the variable power supplies 24 and 34.

In the gas sensor 300, the pump control part 392 of the control part 391 is configured to control the operation of the oxygen pump cell 21 and the hydrogen pump cell 31 so as to measure a concentration of a target gas to be measured (in this embodiment, each of oxygen and water vapor) in a measurement-object gas.

In the gas sensor 300, the pump control part 392 applies a predetermined pump voltage Vp0 between the intracavity oxygen pump electrode (the inner oxygen pump electrode 22) and the extracavity oxygen pump electrode (the outer oxygen pump electrode 23) in the oxygen pump cell 21 based on an electromotive force V0 between the reference electrode 42 and the intracavity oxygen pump electrode (the inner oxygen pump electrode 22) of the oxygen pump cell 21 (namely, the electromotive force V0 in the oxygen pump controlling sensor cell 80) to make an oxygen pump current Ip0 flow through the oxygen pump cell 21; and applies a predetermined pump voltage Vp1 between the intracavity hydrogen pump electrode (the inner hydrogen pump electrode 32) and the extracavity hydrogen pump electrode (the outer hydrogen pump electrode 33) in the hydrogen pump cell 31 to make a hydrogen pump current Ip1 flow through the hydrogen pump cell 31.

In the gas sensor 300, the pump control part 392 performs feedback control of the pump voltage Vp0 of the variable power supply 24 in the oxygen pump cell 21 so that the electromotive force V0 in the oxygen pump controlling sensor cell 80 is at a predetermined target value (referred to as a target value V0_{SET}). The electromotive force V0 indicates the oxygen partial pressure in the vicinity of the inner oxygen pump electrode 22, and therefore making the electromotive force V0 constant means that the oxygen partial pressure in the vicinity of the inner oxygen pump electrode 22 is made constant. As a result, the oxygen pump current Ip0 in the oxygen pump cell 21 varies depending on a total amount (a total concentration) of oxygen gas in the measurement-object gas and oxygen generated by decomposition of water vapor.

The target value V0_{SET} may be appropriately set as a value such that all or substantially all of water vapor in the measurement-object gas is decomposed in the first internal cavity 20 (especially, around the inner oxygen pump electrode 22). A value of the target value V0_{SET} may vary depending on the intended use of the gas sensor 300, the configuration of the sensor element 301 and the like, and the target value V0_{SET} may be, for example, about 800 mV or more and about 1500 mV or less, or, about 1000 mV or more and about 1500 mV or less. As a result of the feedback control, the pump voltage Vp0 may be, for example, about 800 mV or more and about 1500 mV or less, or, about 1000 mV or more and about 1500 mV or less.

In the gas sensor 300, as in the case of the pump control part 92, the pump control part 392 applies, between the intracavity hydrogen pump electrode (the inner hydrogen pump electrode 32) and the extracavity hydrogen pump electrode (the outer hydrogen pump electrode 33) in the hydrogen pump cell 31, such a predetermined pump voltage Vp1 that all or substantially all of hydrogen in the measurement-object gas introduced into the second internal cavity 40 is pumped out from the second internal cavity 40 (especially, around the inner hydrogen pump electrode 32). The pump voltage Vp1 may vary depending on the intended use of the gas sensor 300, the configuration of the sensor element 301 and the like, and the pump voltage Vp1 may be, for example, about 100 mV or more and about 500 mV or less.

Accordingly, when the pump control part 392 performs the feedback control of the pump voltage Vp0 of the variable power supply 24 in the oxygen pump cell 21, all or substantially all of water vapor in the measurement-object gas can be decomposed at the inner oxygen pump electrode 22 of the oxygen pump cell 21 more surely. An amount of the hydrogen generated by the decomposition corresponds to the water vapor concentration in the measurement-object gas with higher accuracy, and therefore, the hydrogen pump current Ip1 flowing through the hydrogen pump cell 31 can become at a current value corresponding to the water vapor concentration in the measurement-object gas with higher accuracy. Further, since oxygen is pumped out by the oxygen pump cell 21 more accurately, the oxygen pump current Ip0 can become a value corresponding to the total amount of the oxygen gas in the measurement-object gas and the oxygen generated by the decomposition of water vapor with higher accuracy. As a result, water vapor concentration and oxygen concentration in the measurement-object gas can be detected with much higher accuracy.

### Embodiment 4

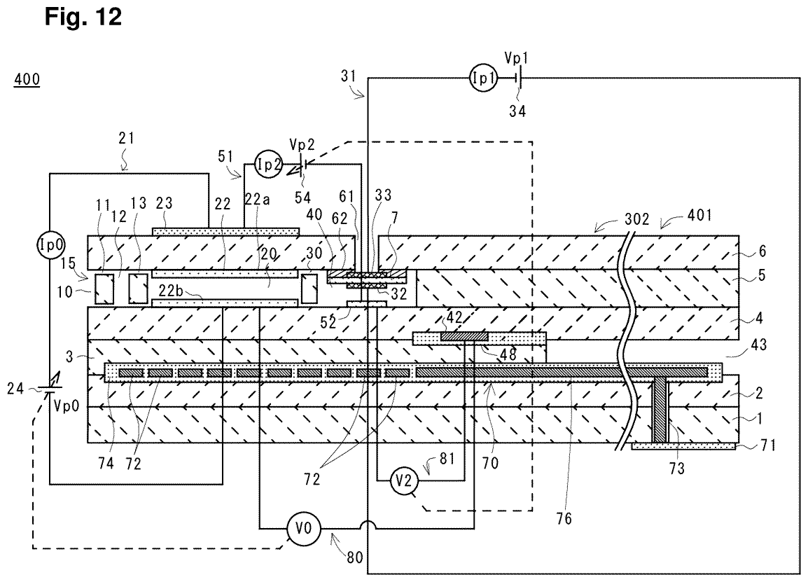

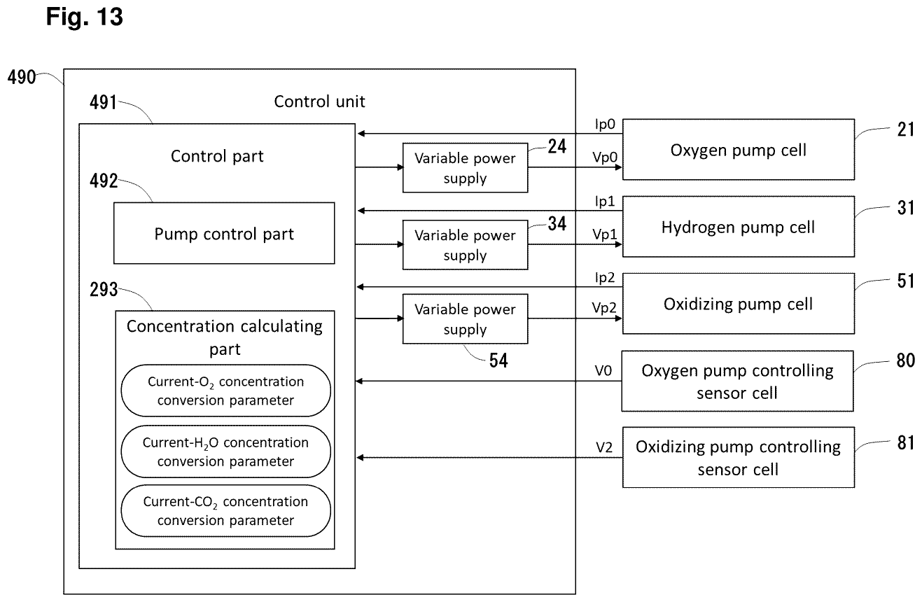

As a gas sensor 400 of Embodiment 4, an example including a sensor element 401 having a reference electrode 42 is shown. The gas sensor 400 of Embodiment 4 is, as in the case of the gas sensor 200 of Embodiment 2, an example of a gas sensor that detects oxygen O₂, water vapor H₂O, and carbon dioxide CO₂ in a measurement-object gas, and measures the concentrations of O₂, H₂O and CO₂. FIG. 12 is a vertical sectional schematic view in the longitudinal direction of the sensor element 401, showing one example of a schematic configuration of the gas sensor 400 of Embodiment 4. In FIG. 12, the same member as in FIG. 5 or 10 is denoted by the same sign. FIG. 13 is a block diagram showing electric connections between the control unit 490 and the sensor element 401 in the gas sensor 400 of Embodiment 4.

In the sensor element 401, the arrangement of the measurement-object gas flow cavity 15 and each of the electrodes is the same as in the case of the sensor element 201 in the above-described gas sensor 200 of Embodiment 2.

In the sensor element 401, the reference gas introduction space 43, the air introduction layer 48 and the reference electrode 42 are provided as in the case of the sensor element 301.

In the sensor element 401, the reference electrode 42 electrochemically corresponds to the intracavity oxygen pump electrode (the inner oxygen pump electrode 22), as in the case of the sensor element 301. That is, the reference electrode 42 is disposed adjacent to the intracavity oxygen pump electrode (the inner oxygen pump electrode 22) via the oxygen-ion-conductive solid electrolyte layer (in this case, the second oxygen-ion conductor layer 6, the spacer layer 5, and the first oxygen-ion conductor layer 4).

Specifically, the inner oxygen pump electrode 22, the second oxygen-ion conductor layer 6, the spacer layer 5, the first oxygen-ion conductor layer 4, and the reference electrode 42 form an electrochemical sensor cell, namely, an oxygen pump controlling sensor cell 80. The oxygen concentration (oxygen partial pressure) in the first internal cavity 20 can be detected from an electromotive force V0 measured between the inner oxygen pump electrode 22 and the reference electrode 42 in the oxygen pump controlling sensor cell 80.

In addition, a control is performed to apply the pump voltage Vp0 in the oxygen pump cell 21 based on the electromotive force V0. More specifically, the oxygen pump current Ip0 is controlled by performing feedback control of the pump voltage Vp0 so that the electromotive force V0 is constant. Thus, the oxygen concentration in the first internal cavity 20 can be controlled at a predetermined concentration, and it is therefore possible to more surely decompose substantially all of water vapor and carbon dioxide in the measurement-object gas and pump out oxygen in the first internal cavity 20. Therefore, water vapor concentration, carbon dioxide concentration and oxygen concentration in the measurement-object gas can be detected with higher accuracy.

In the sensor element 401, the reference electrode 42 also electrochemically corresponds to the intracavity oxidizing electrode (the inner oxidizing electrode 52). That is, the reference electrode 42 is disposed adjacent to the intracavity oxidizing electrode (the inner oxidizing electrode 52) via the oxygen-ion-conductive solid electrolyte layer (in this case, the first oxygen-ion conductor layer 4).

Specifically, the inner oxidizing electrode 52, the first oxygen-ion conductor layer 4, and the reference electrode 42 form an electrochemical sensor cell, namely, an oxidizing pump controlling sensor cell 81. The oxygen concentration (oxygen partial pressure) in the second internal cavity 40 (especially, in the vicinity of the inner oxidizing electrode 52) can be detected from an electromotive force V2 measured between the inner oxidizing electrode 52 and the reference electrode 42 in the oxidizing pump controlling sensor cell 81.

In addition, a control is performed to apply the pump voltage Vp2 in the oxidizing pump cell 51 based on the electromotive force V2. More specifically, the oxidizing pump current Ip2 is controlled by performing feedback control of the pump voltage Vp2 so that the electromotive force V2 is constant. Thus, the oxygen concentration in the second internal cavity 40 can be controlled at a predetermined concentration, and it is therefore possible to more accurately oxidize, at the inner oxidizing electrode 52, substantially all of carbon monoxide generated by the decomposition of the carbon dioxide in the first internal cavity 20. Therefore, carbon dioxide concentration in the measurement-object gas can be detected with higher accuracy.

FIG. 13 is a block diagram showing electric connections between the control unit 490 and the respective pump cells 21, 31 and 51 and the respective sensor cells 80 and 81 of the sensor element 401 in the gas sensor 400 of Embodiment 4. In FIG. 13, the same member as in FIG. 7 is denoted by the same sign. The control unit 490 includes the variable power supplies 24, 34 and 54, and a control part 491. The control part 491 includes a pump control part 492 and the concentration calculating part 293.

The control part 491 is configured to acquire a pump current (Ip0, Ip1, Ip2) in each of the pump cells 21, 31 and 51, and the electromotive force (V0, V2) in each of the sensor cells 80 and 81 of the sensor element 401. Further, the control part 491 is configured to output control signals to the variable power supplies 24, 34 and 54.

In the gas sensor 400, the pump control part 492 of the control part 491 is configured to control the oxygen pump cell 21, the hydrogen pump cell 31 and the oxidizing pump cell 51 so as to measure a concentration of a target gas to be measured (in this embodiment, each of oxygen O₂, water vapor H₂O, and carbon dioxide CO₂) in a measurement-object gas.

The pump control part 492 applies a predetermined pump voltage Vp0 between the intracavity oxygen pump electrode (the inner oxygen pump electrode 22) and the extracavity oxygen pump electrode (the outer oxygen pump electrode 23) in the oxygen pump cell 21 based on an electromotive force V0 between the reference electrode 42 and the intracavity oxygen pump electrode (the inner oxygen pump electrode 22) of the oxygen pump cell 21 (namely, the electromotive force V0 in the oxygen pump controlling sensor cell 80) to make an oxygen pump current Ip0 flow through the oxygen pump cell 21; applies a predetermined pump voltage Vp1 between the intracavity hydrogen pump electrode (the inner hydrogen pump electrode 32) and the extracavity hydrogen pump electrode (the outer hydrogen pump electrode 33) in the hydrogen pump cell 31 to make a hydrogen pump current Ip1 flow through the hydrogen pump cell 31; and applies a predetermined pump voltage Vp2 between the intracavity oxidizing electrode (the inner oxidizing electrode 52) and the extracavity oxidizing electrode (the outer oxygen pump electrode 23) in the oxidizing pump cell 51 based on an electromotive force V2 between the reference electrode 42 and the intracavity oxidizing electrode (the inner oxidizing electrode 52) in the oxidizing pump cell 51 (namely, the electromotive force V2 in the oxidizing pump controlling sensor cell 81) to make an oxidizing pump current Ip2 flow through the oxidizing pump cell 51.

In the gas sensor 400, the pump control part 492 performs feedback control of the pump voltage Vp0 of the variable power supply 24 in the oxygen pump cell 21 so that the electromotive force V0 in the oxygen pump controlling sensor cell 80 is at a predetermined target value (referred to as a target value V0_{SET}). The electromotive force V0 indicates the oxygen partial pressure in the vicinity of the inner oxygen pump electrode 22, and therefore making the electromotive force V0 constant means that the oxygen partial pressure in the vicinity of the inner oxygen pump electrode 22 is made constant. As a result, the oxygen pump current Ip0 in the oxygen pump cell 21 varies depending on a total amount (a total concentration) of oxygen gas in the measurement-object gas, oxygen generated by decomposition of water vapor, and oxygen generated by decomposition of carbon dioxide.

The target value V0_{SET} may be appropriately set as a value such that all or substantially all of water vapor and carbon dioxide in the measurement-object gas is decomposed in the first internal cavity 20 (especially, around the inner oxygen pump electrode 22). A value of the target value V0_{SET} may vary depending on the intended use of the gas sensor 400, the configuration of the sensor element 401 and the like, and the target value V0_{SET} may be, for example, about 800 mV or more and about 1500 mV or less, or, about 1000 mV or more and about 1500 mV or less. As a result of the feedback control, the pump voltage Vp0 may be, for example, about 800 mV or more and about 1500 mV or less, or, about 1000 mV or more and about 1500 mV or less.

In the gas sensor 400, as in the case of the pump control part 92, the pump control part 492 applies, between the intracavity hydrogen pump electrode (the inner hydrogen pump electrode 32) and the extracavity hydrogen pump electrode (the outer hydrogen pump electrode 33) in the hydrogen pump cell 31, such a predetermined pump voltage Vp1 that all or substantially all of hydrogen in the measurement-object gas introduced into the second internal cavity 40 is pumped out from the second internal cavity 40 (especially, around the inner hydrogen pump electrode 32). The pump voltage Vp1 may vary depending on the intended use of the gas sensor 400, the configuration of the sensor element 401 and the like, and the pump voltage Vp1 may be, for example, about 100 mV or more and about 500 mV or less.

In the gas sensor 400, the pump control part 492 performs feedback control of the pump voltage Vp2 of the variable power supply 54 in the oxidizing pump cell 51 so that the electromotive force V2 in the oxidizing pump controlling sensor cell 81 is at a predetermined target value (referred to as a target value V2_{SET}). The electromotive force V2 indicates the oxygen partial pressure in the vicinity of the inner oxidizing electrode 52, and therefore making the electromotive force V2 constant means that the oxygen partial pressure in the vicinity of the inner oxidizing electrode 52 is made constant. As a result, the oxidizing pump current Ip2 in the oxidizing pump cell 51 varies depending on a total amount (a total concentration) of the carbon monoxide generated by the decomposition of the carbon dioxide in the measurement-object gas.

The target value V2_{SET} may be appropriately set as a value such that all or substantially all of carbon monoxide in the measurement-object gas introduced into the second internal cavity 40 is oxidized in the second internal cavity 40 (especially, around the inner oxidizing electrode 52). A value of the target value V2_{SET} may vary depending on the intended use of the gas sensor 400, the configuration of the sensor element 401 and the like, and the target value V2_{SET} may be, for example, about 100 mV or more and about 200 mV or less. As a result of the feedback control, the pump voltage Vp2 may be, for example, about 100 mV or more and about 200 mV or less.

Accordingly, when the pump control part 492 performs the feedback control of the pump voltage Vp0 of the variable power supply 24 in the oxygen pump cell 21, all or substantially all of water vapor and carbon dioxide in the measurement-object gas can be decomposed at the inner oxygen pump electrode 22 of the oxygen pump cell 21 more surely. An amount of the hydrogen generated by the decomposition of the water vapor corresponds to the water vapor concentration in the measurement-object gas with higher accuracy, and therefore, the hydrogen pump current Ip1 flowing through the hydrogen pump cell 31 can become at a current value corresponding to the water vapor concentration in the measurement-object gas with higher accuracy. An amount of the carbon monoxide generated by the decomposition of the carbon dioxide corresponds to the carbon dioxide concentration in the measurement-object gas with higher accuracy, and therefore, the oxidizing pump current Ip2 flowing through the oxidizing pump cell 51 can become at a current value corresponding to the carbon dioxide concentration in the measurement-object gas with higher accuracy. Further, since oxygen is pumped out by the oxygen pump cell 21 more accurately, the oxygen pump current Ip0 can become at a value corresponding to the total amount of the oxygen gas in the measurement-object gas, the oxygen generated by the decomposition of water vapor, and the oxygen generated by the decomposition of carbon dioxide with higher accuracy. As a result, water vapor concentration, carbon dioxide concentration, and oxygen concentration in the measurement-object gas can be detected with much higher accuracy.

Embodiments 1 to 4 have been described above as examples of the embodiments according to the present invention, but the present invention is not limited thereto. The present invention may include a gas sensor having any structure including a sensor element and a control unit as long as the object of the present invention can be achieved, that is, a gas sensor capable of measuring a target gas to be measured in a measurement-object gas with high accuracy is provided.

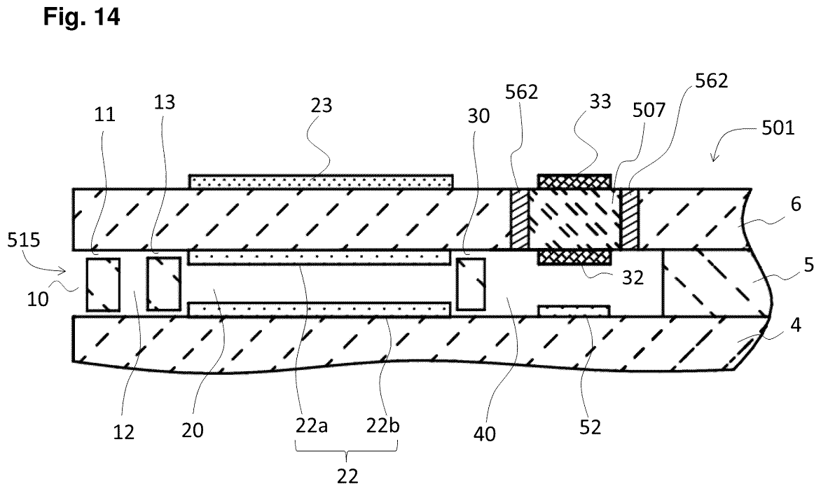

In the above-described Embodiments 1 to 4, the proton conductor layer 7 is disposed to cover the penetrating hole 61 made in the second oxygen-ion conductor layer 6, but the present invention is not limited thereto. For example, the proton conductor layer may be embedded in a part of the second oxygen-ion conductor layer 6. FIG. 14 is a partial sectional schematic view of a sensor element 501, showing an embodiment with different arrangement of the proton conductor layer. FIG. 14 is the same partial section of FIG. 6. It is to be noted that the same configuration may be possible with or without the inner oxidizing electrode 52 and/or the reference electrode 42.

In the sensor element 501, in a plane including a longitudinal direction of the sensor element 501 and a width direction perpendicular to the longitudinal direction, that is, in a horizontal plane, a proton conductor layer 507 and the second oxygen-ion conductor layer 6 are present on the same plane. In other words, the second oxygen-ion conductor layer 6 and the proton conductor layer 507 have substantially the same in thickness, and exist on the same plane. The second oxygen-ion conductor layer 6 is hollowed out so as to penetrate a portion of the second oxygen-ion conductor layer 6 in a thickness direction at a position facing the second internal cavity 40, and the proton conductor layer 507 exists in the portion hollowed out. An insulator layer 562 is interposed between the second oxygen-ion conductor layer 6 and the proton conductor layer 507 so that the second oxygen-ion conductor layer 6 and the proton conductor layer 507 are electrically insulated from each other. The inner hydrogen pump electrode 32 is disposed facing the second internal cavity 40 on a lower surface of the proton conductor layer 507, and the outer hydrogen pump electrode 33 is disposed on an upper surface of the proton conductor layer 507. The proton conductor layer 507 may occupy the whole in the width direction of the sensor element 501, or a part of the width. A planar shape of the proton conductor layer 507 may be a circular shape, an elliptical shape, a rectangular shape or the like.

As in the case of the above-described Embodiments 1 to 4, both of the second oxygen-ion conductor layer 6 and the proton conductor layer 7 are preferably present on the ceiling surface (namely, one surface in inner surfaces of the internal cavity) of the internal cavity. The proton conductor layer 7 may be present at a portion where the hydrogen pump cell 31 is to be formed. Since the proton conductor layer 7 need not be formed as a layer extending the entire length of the sensor element in the longitudinal direction, the manufacturing process of the sensor element can be simplified.

### [Method for Producing Gas Sensor]

Next, one example of a method for producing the gas sensor as described above is described. A plurality of unfired sheet moldings (so-called green sheets) containing an oxygen-ion-conductive solid electrolyte such as zirconia (ZrO₂) as a ceramic component are subjected to a predetermined processing and printing of circuit pattern, and then the plurality of sheets are laminated, and the laminate was cut, and then fired. Thus the sensor element 101 can be manufactured. Then, the manufactured sensor element may be incorporated into the gas sensor.

Hereinafter, description is made while taking the case of manufacturing the sensor element 101 composed of six layers shown in FIG. 1 as an example. It is to be noted that the gas sensors 110, 200, 300, and 400 shown in FIGS. 4, 5, 8, and 10, respectively, can also be manufactured in the same manner.

First, six green sheets containing an oxygen-ion-conductive solid electrolyte such as zirconia (ZrO₂) as a ceramic component are prepared. For manufacturing of the green sheets, a known molding method can be used. The six green sheets may all have the same thickness, or the thickness differs depending on the layer to be formed. In each of the six green sheets, sheet holes or the like for use in positioning at the time of printing or stacking are formed in advance by a known method such as a punching process with a punching apparatus to prepare a blank sheet. In the blank sheet for use as the spacer layer 5, penetrating parts such as internal cavities are also formed in the same manner. In the blank sheet for use as the second oxygen-ion conductor layer 6, a penetrating part to be the penetrating hole 61 is also formed in the same manner. Also in the remaining layers, necessary penetrating parts are formed in advance.

The blank sheets for use as six layers, namely, the first substrate layer 1, the second substrate layer 2, the third substrate layer 3, the first oxygen-ion conductor layer 4, the spacer layer 5, and the second oxygen-ion conductor layer 6 are subjected to printing of various patterns required for respective layers and drying treatment. For printing of a pattern, a known screen printing technique can be used. Also as the drying treatment, a known drying means can be used.

For example, the proton conductor layer 7 may be formed by screen printing. Since formation of a green sheet of the proton conductor is not required, the manufacturing process can be simplified. First, an insulator layer paste used to form the insulator layer 62, an electrode paste used to form the inner hydrogen pump electrode 32 and the outer hydrogen pump electrode 33, and a proton conductor paste used to form the proton conductor layer 7 are prepared. Each of the pastes is prepared by blending a raw material powder composed of the material of an object to be formed, and an organic binder, an organic solvent, etc.

Then, the insulator layer paste is printed in a desired pattern on the second oxygen-ion conductor layer 6 at a position where the insulator layer 62 is to be formed. The electrode paste is printed in a desired pattern at a position where the outer hydrogen pump electrode 33 is to be formed. The proton conductor paste is printed in a desired pattern at a position where the proton conductor layer 7 is to be formed. The electrode paste is printed in a desired pattern at a position where the inner hydrogen pump electrode 32 is to be formed.

After completing the printing and drying of diverse patterns for each of the six blank sheets by repeating these steps, contact bonding treatment of stacking the six printed blank sheets in a predetermined order while positioning with the sheet holes and the like, and contact bonding at a predetermined temperature and pressure condition to give a laminate is conducted. The contact bonding treatment is conducted by heating and pressurizing with a known laminator such as a hydraulic press. While the temperature, the pressure and the time of heating and pressurizing depend on the laminator being used, they may be appropriately determined to achieve excellent lamination.

For example, the proton conductor layer 7 may be prepared as a green sheet, as in the case of the second oxygen-ion conductor layer 6. In this case, in the above contact bonding treatment, the proton conductor layer 7 may be stacked in a desired position and contact bonded.

The obtained laminate includes a plurality of sensor elements 101. The laminate is cut into units of the sensor element 101. The cut laminate is fired at a predetermined firing temperature to obtain the sensor element 101. That is, the sensor element 101 is obtained by integral firing (co-firing) of the solid electrolyte layers and the electrodes. The firing temperature may be such a temperature that the solid electrolyte forming the base part 102 of the sensor element 101 is sintered to become a dense product, and an electrode or the like maintains desired porosity. The firing is conducted, for example, at a firing temperature of about 1200° C. or more and 1500° C. or less.

The obtained sensor element 101 is incorporated into the gas sensor 100 in such a form that the front end part of the sensor element 101 comes into contact with the measurement-object gas, and the rear end part of the sensor element 101 comes into contact with the reference gas.

### EXAMPLES

Hereinafter, explanation will be made using Examples. The present invention is not limited to the following Examples.

As Example 1, the gas sensor 400 of Embodiment 4 shown in FIG. 12 was produced in accordance with the above-described production method of the gas sensor.

For the produced gas sensor 400, change in each of the pump currents Ip0, Ip1, and Ip2 was evaluated when oxygen concentration, water vapor concentration and carbon dioxide concentration were varied. Specifically, the gas sensor 400 was attached to a piping for measurement, and was driven. That is, the sensor element 401 was heated by the heater 72 and kept at a driving temperature (about 800° C.), and the pump control part 492 was performed the above-described control. The control was performed by setting the target value V0_{SET} to 1300 mV, the pump voltage Vp1 to 400 mV, and the target value V2_{SET} to 200 mV. A model gas having a predetermined gas composition was made to flow in the piping for measurement, and each of the pump current Ip0, Ip1 and Ip2 was measured.

Each of the pump current Ip0, Ip1 and Ip2 was measured when oxygen concentration was varied. The composition of the model gas was as follows. Each “%” means % by volume (the same hereinafter).
Oxygen concentration: 0, 5, 10 and 20%;
Water vapor concentration: 3%;
Carbon dioxide concentration: 0%; and
Other gas component: Nitrogen (reminder).

Each of the pump current Ip0, Ip1 and Ip2 was measured when water vapor concentration was varied. The composition of the model gas was as follows.
Oxygen concentration: 10%;
Water vapor concentration: 3, 5, 10 and 20%;
Carbon dioxide concentration: 0%; and
Other gas component: Nitrogen (reminder).

Each of the pump current Ip0, Ip1 and Ip2 was measured when carbon dioxide concentration was varied. The composition of the model gas was as follows.
Oxygen concentration: 10%;
Water vapor concentration: 3%;
Carbon dioxide concentration: 0, 2, 6, 10, 15, and 20%; and
Other gas component: Nitrogen (reminder).

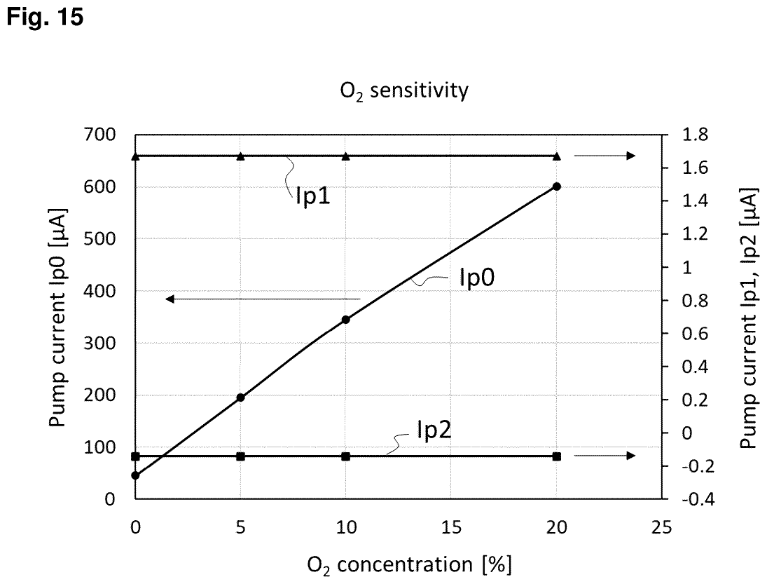

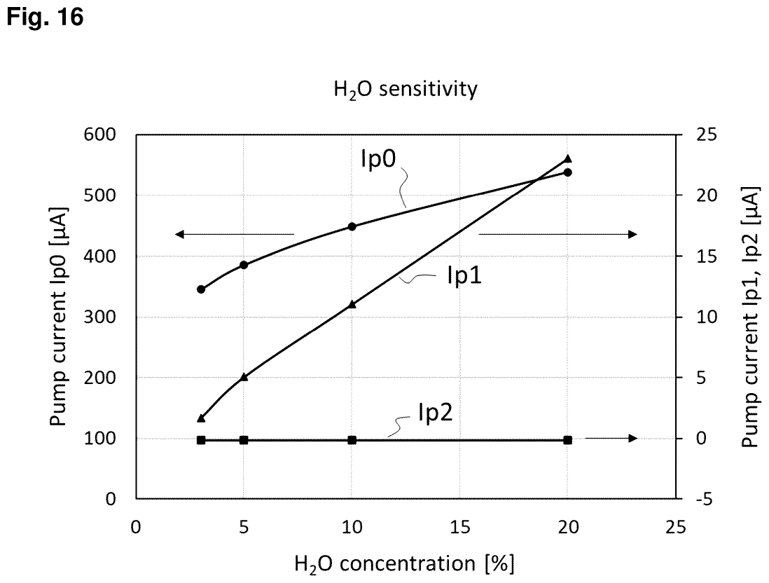

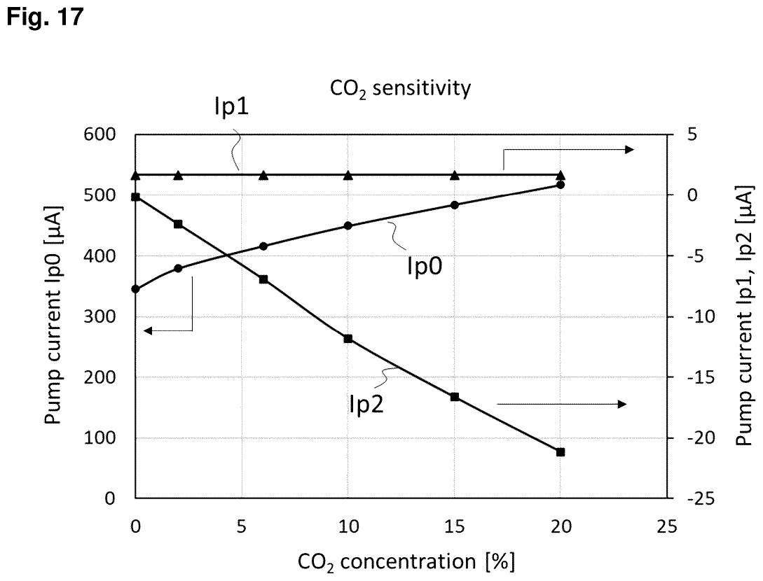

FIG. 15 to FIG. 17 shows each of measurement results. FIG. 15 is a graph showing a relationship (namely, O₂ sensitivity) between the oxygen concentration in the measurement-object gas (or, the model gas) and each of the pump current Ip0, Ip1 and Ip2. FIG. 16 is a graph showing a relationship (namely, H₂O sensitivity) between the water vapor concentration in the measurement-object gas (or, the model gas) and each of the pump current Ip0, Ip1 and Ip2. FIG. 17 is a graph showing a relationship (namely, CO₂ sensitivity) between the carbon dioxide concentration in the measurement-object gas (or, the model gas) and each of the pump current Ip0, Ip1 and Ip2. In each of FIG. 15 to FIG. 17, the vertical axis represents the oxygen pump current Ip0 (μA) for the primary axis, and the hydrogen pump current Ip1 and the oxidizing pump current Ip2 (μA) for the secondary axis. In FIG. 15 to FIG. 17, the horizontal axis represents the oxygen concentration (%), the water vapor concentration (%), and the carbon dioxide concentration (%), respectively.

As shown in FIG. 15 to FIG. 17, it was confirmed that the hydrogen pump current Ip1 increased linearly with increasing the water vapor concentration (see FIG. 16), but did not change with increasing the oxygen concentration or the carbon dioxide concentration (see FIG. 15 and FIG. 17). Accordingly, it was confirmed that the water vapor concentration in the measurement-object gas can be measured based on the hydrogen pump current Ip1 in the gas sensor 400 of Example 1.

As shown in FIG. 15 to FIG. 17, it was confirmed that the oxidizing pump current Ip2 decreased linearly (namely, an absolute value of a current value of the oxygen pump current Ip0 increased linearly) with increasing the carbon dioxide concentration (see FIG. 17), but did not change with increasing the oxygen concentration or the water vapor concentration (see FIG. 15 and FIG. 16). Accordingly, it was confirmed that the carbon dioxide concentration in the measurement-object gas can be measured based on the oxidizing pump current Ip2 in the gas sensor 400 of Example 1.

As shown in FIG. 15 to FIG. 17, it was confirmed that the oxygen pump current Ip0 changed in its current value with respect to change in each of the oxygen concentration, the water vapor concentration and the carbon dioxide concentration. It was therefore confirmed that the oxygen concentration in the measurement-object gas can be measured based on the oxygen pump current Ip0, the hydrogen pump current Ip1 and the oxidizing pump current Ip2 as described above.

As described above, according to the present invention, by having a solid electrolyte layer containing a plurality of solid electrolytes, in other words, by combining an oxygen-ion conductor layer and a proton conductor layer, it is possible to provide a gas sensor that can measure a target gas to be measured in a measurement-object gas with high accuracy. For example, an O₂/CO₂/H₂O sensor can be provided.

### EXPLANATION OF REFERENCE SIGNS IN THE DRAWINGS

1: first substrate layer; 2: second substrate layer; 3: third substrate layer; 4: first oxygen-ion conductor layer; 5: spacer layer; 6: second oxygen-ion conductor layer; 7: proton conductor layer; 10: gas inlet; 11: first diffusion-rate limiting path; 12: buffer space; 13: fourth diffusion-rate limiting path; 14, 15, 16, 17, 515: measurement-object gas flow cavity; 20: first internal cavity; 21: oxygen pump cell; 22: inner oxygen pump electrode; 22a: ceiling electrode portion (of the inner oxygen pump electrode); 22b: bottom electrode portion (of the inner oxygen pump electrode); 23: outer oxygen pump electrode; 24: variable power supply (of the oxygen pump cell); 25: internal cavity; 30: second diffusion-rate limiting path; 31: hydrogen pump cell; 32: inner hydrogen pump electrode; 33: outer hydrogen pump electrode; 34: variable power supply (of the hydrogen pump cell); 40: second internal cavity; 42: reference electrode; 43: reference gas introduction space; 48: air introduction layer; 51: oxidizing pump cell; 52: inner oxidizing electrode; 54: variable power supply (of the oxidizing pump cell); 60: third diffusion-rate limiting path; 61: penetrating hole; 62: insulator layer; 65: third internal cavity; 70: heater part; 71: heater electrode; 72: heater; 73: through hole; 74: heater insulating layer; 76: heater lead; 80: oxygen pump controlling sensor cell; 81: oxidizing pump controlling sensor cell; 90, 290, 390, 490: control unit; 91, 291, 391, 491: control part; 92, 292, 392, 492: pump control part; 93, 293: concentration calculating part; 100, 110, 200, 300, 400: gas sensor; 101, 111, 201, 211, 221, 301, 401, 501: sensor element; and 102, 112, 302: base part.

What is claimed is:

1. A gas sensor for detecting a target gas to be measured in a measurement-object gas, the gas sensor comprising a sensor element and a control unit for controlling the sensor element, wherein
the sensor element comprises:
a base part in an elongated plate shape, including an oxygen-ion-conductive solid electrolyte layer and a proton-conductive solid electrolyte layer, and having an insulator layer interposed between the oxygen-ion-conductive solid electrolyte layer and the proton-conductive solid electrolyte layer;
a measurement-object gas flow cavity having a gas inlet that opens on a surface of the base part; and an internal cavity that communicates with the gas inlet via a first diffusion-rate limiting path, on an inner surface of the internal cavity the oxygen-ion-conductive solid electrolyte layer and the proton-conductive solid electrolyte layer being present;
an oxygen pump cell including: an intracavity oxygen pump electrode disposed on the oxygen-ion-conductive solid electrolyte layer in the internal cavity of the measurement-object gas flow cavity; and an extracavity oxygen pump electrode disposed at a position different from the measurement-object gas flow cavity on the base part and adjacent to the intracavity oxygen pump electrode via the oxygen-ion-conductive solid electrolyte layer; and
a hydrogen pump cell including: an intracavity hydrogen pump electrode disposed on the proton-conductive solid electrolyte layer in the internal cavity of the measurement-object gas flow cavity; and an extracavity hydrogen pump electrode disposed at a position different from the measurement-object gas flow cavity on the base part and adjacent to the intracavity hydrogen pump electrode via the proton-conductive solid electrolyte layer, and
the control unit comprises:
a pump control part for controlling operation of the oxygen pump cell and the hydrogen pump cell; and
a concentration calculating part for calculating a concentration of a target gas to be measured in a measurement-object gas, wherein
the pump control part applies a predetermined voltage between the intracavity oxygen pump electrode and the extracavity oxygen pump electrode of the oxygen pump cell to make an oxygen pump current flow through the oxygen pump cell; and applies a predetermined voltage between the intracavity hydrogen pump electrode and the extracavity hydrogen pump electrode of the hydrogen pump cell to make a hydrogen pump current flow through the hydrogen pump cell; and
the concentration calculating part calculates the concentration of the target gas to be measured in the measurement-object gas based on at least one of the oxygen pump current and the hydrogen pump current.

2. The gas sensor according to claim 1, wherein the extracavity oxygen pump electrode and/or the extracavity hydrogen pump electrode are disposed at a position in contact with the measurement-object gas.

3. The gas sensor according to claim 1, wherein the internal cavity has a first internal cavity on an inner surface of which at least the oxygen-ion-conductive solid electrolyte layer is present; and a second internal cavity that communicates with the first internal cavity via a second diffusion-rate limiting path, on an inner surface of the second internal cavity at least the proton-conductive solid electrolyte layer being present, and
the intracavity oxygen pump electrode is disposed in the first internal cavity and the intracavity hydrogen pump electrode is disposed in the second internal cavity.

4. The gas sensor according to claim 1, wherein
the sensor element further comprises:
an oxidizing pump cell including: an intracavity oxidizing electrode disposed at a position farther from the first diffusion-rate limiting path than the intracavity oxygen pump electrode on the oxygen-ion-conductive solid electrolyte layer in the internal cavity of the measurement-object gas flow cavity; and an extracavity oxidizing electrode disposed at a position different from the measurement-object gas flow cavity on the base part and adjacent to the intracavity oxidizing electrode via the oxygen-ion-conductive solid electrolyte layer,
the pump control part further applies a predetermined voltage between the intracavity oxidizing electrode and the extracavity oxidizing electrode of the oxidizing pump cell to make an oxidizing pump current flow through the oxidizing pump cell, and
the concentration calculating part calculates the concentration of the target gas to be measured in the measurement-object gas based on at least one of the oxygen pump current, the hydrogen pump current, and the oxidizing pump current.

5. The gas sensor according to claim 4, wherein the intracavity oxidizing electrode is disposed on the oxygen-ion-conductive solid electrolyte layer at a position farther from the first diffusion-rate limiting path than the intracavity hydrogen pump electrode.

6. The gas sensor according to claim 4, wherein the internal cavity has a first internal cavity on an inner surface of which at least the oxygen-ion-conductive solid electrolyte layer is present; and a second internal cavity that communicates with the first internal cavity via a second diffusion-rate limiting path, on an inner surface of the second internal cavity the oxygen-ion-conductive solid electrolyte layer and the proton-conductive solid electrolyte layer being present, and
the intracavity oxygen pump electrode is disposed in the first internal cavity, the intracavity hydrogen pump electrode is disposed on the proton-conductive solid electrolyte layer in the second internal cavity, and the intracavity oxidizing electrode is disposed on the oxygen-ion-conductive solid electrolyte layer in the second internal cavity.

7. The gas sensor according to claim 4, wherein the internal cavity has a first internal cavity on an inner surface of which at least the oxygen-ion-conductive solid electrolyte layer is present; a second internal cavity that communicates with the first internal cavity via a second diffusion-rate limiting path, on an inner surface of the second internal cavity at least the proton-conductive solid electrolyte layer being present; and a third internal cavity that communicates with the second internal cavity via a third diffusion-rate limiting path, on an inner surface of the third internal cavity at least the oxygen-ion-conductive solid electrolyte layer being present, and
the intracavity oxygen pump electrode is disposed in the first internal cavity, the intracavity hydrogen pump electrode is disposed in the second internal cavity, and the intracavity oxidizing electrode is disposed in the third internal cavity.

8. The gas sensor according to claim 1, wherein
the sensor element comprises a reference electrode disposed inside the base part to be in contact with a reference gas, and
the pump control part applies the predetermined voltage between the intracavity oxygen pump electrode and the extracavity oxygen pump electrode of the oxygen pump cell based on an electromotive force between the reference electrode and the intracavity oxygen pump electrode of the oxygen pump cell to make the oxygen pump current flow through the oxygen pump cell.

9. The gas sensor according to claim 1, wherein, in a plane including a longitudinal direction of the base part and a width direction perpendicular to the longitudinal direction, the proton-conductive solid electrolyte layer and the oxygen-ion-conductive solid electrolyte layer are present on the same plane, and the insulator layer is interposed between the oxygen-ion-conductive solid electrolyte layer and the proton-conductive solid electrolyte layer.

10. The gas sensor according to claim 1, wherein
the oxygen-ion-conductive solid electrolyte layer has a penetrating hole,
the proton-conductive solid electrolyte layer is parallel to the oxygen-ion-conductive solid electrolyte layer, and covers the penetrating hole on a surface of the oxygen-ion-conductive solid electrolyte layer facing the internal cavity; and the insulator layer is interposed between the proton-conductive solid electrolyte layer and the oxygen-ion-conductive solid electrolyte layer,
the intracavity hydrogen pump electrode is disposed on a surface of the proton-conductive solid electrolyte layer facing the internal cavity, and
the extracavity hydrogen pump electrode is disposed at a position corresponding to the penetrating hole on a surface of the proton-conductive solid electrolyte layer opposite to the surface on which the intracavity hydrogen pump electrode is disposed.

11. The gas sensor according to claim 1, wherein the target gas to be measured is at least one selected from the group consisting of oxygen and water vapor.

12. The gas sensor according to claim 4, wherein the target gas to be measured is at least one selected from the group consisting of oxygen, water vapor and carbon dioxide.
